(19) United States
(12) Patent Application Publication
(10) Pub. No.: US 2011/0076283 A1
(43) Pub. Date: Mar. 31, 2011

(54) METHOD FOR PREDICTING AND DIAGNOSING BRAIN TUMOR

(75) Inventors: Monika Hegi, Lausanne (CH); Anastasia Murat, Bale (CH); Eugenia Migliavacca, Lausanne (CH); Roger Stupp, Lausanne (CH)

(73) Assignee: Universite De Lausanne, Lausanne (CH)

(21) Appl. No.: 12/864,278

(22) PCT Filed: Jan. 23, 2009
(86) PCT No.: PCT/IB2009/050279

§ 371 Date: Nov. 4, 2010

Related U.S. Application Data
(60) Provisional application No. 61/006,627, filed on Jan. 24, 2008.

(51) Int. Cl. A61K 39/395; C12Q 1/68; C40B 30/04; A61K 31/7088; C07H 21/02; C07K 16/18; A61P 35/00

(52) U.S. Cl. 424/172.1; 435/6; 506/9; 514/44 A; 536/23.1; 536/24.5; 530/389.1

(57) ABSTRACT

The present invention relates to a method for predicting or diagnosing outcome of concomitant chemo-radiotherapy of a subject suffering from brain tumor. The present invention further relates to compositions and methods for treatment or prevention of tumor resistance in a subject suffering from a brain tumor and to a kit useful for predicting or diagnosing the tumor resistance in a subject treated with concomitant chemo-radiotherapy.

### FIELD OF THE INVENTION

The present invention relates to a method for predicting or diagnosing outcome of concomitant chemo-radiotherapy of a subject suffering from brain tumor. The present invention further relates to compositions and methods for treatment or prevention of tumor resistance in a subject suffering from a brain tumor and to a kit useful for predicting or diagnosing the tumor resistance in a subject treated with concomitant chemo-radiotherapy.

### BACKGROUND OF THE INVENTION

Radiotherapy, chemotherapy, or a combination thereof are used to treat human tumors. In glioblastoma, introduction of combined chemo-radiotherapy of concomitant and adjuvant temozolomide (TMZ) and radiotherapy (TMZ/RT→TMZ) has allowed to significantly prolong survival (Stupp et al., 2005), in particular in patients with an epigenetically silenced O-6-methylguanine-DNA methyltransferase (MGMT) DNA repair gene (Hegi et al., 2005). However, outcome remains unsatisfactory and ongoing clinical trials explore modulation of MGMT or the addition of targeted agents. Recognizing molecular tumor signatures of underlying biological processes associated with resistance in patients treated with this new “standard therapy” will allow the identification of potential targets for improvement of therapy and the development of biomarkers for patient selection.

Several gene expression signatures associated with resistance to therapy were identified. Tumor resistance was associated with clustered genes dominated by HOX genes; and with two clusters G25 and G13 reflecting amplification driven overexpression of proto-oncogenes, EGFR on chromosome 7, and CDK4 & MDM2 on chromosome 12, respectively. An additional cluster, G18 associated with brain physiology was correlated with resistance. Good prognosis was associated with clusters reflecting tumor-host interaction-related features comprising tumor stroma, characterized by markers for tumor blood vessels and myeloid lineage markers/cell adhesion, G7 and G14, and innate immune response G24. Most interestingly the cluster dominated by HOX genes was reminescent of a stem cell-related “self-renewal signature”. HOX gene expression is essential for axis determination during embryogenesis. Studies have reported deregulated HOX gene expression (defined as expression of normal HOX genes in a wrong cellular context) in a variety of cancers as shown in in vitro and in vivo mouse models (Abate-Shen, 2002). Similarly, in glioma, increased expression of HOX genes, as compared to normal brain, has been reported from astrocytoma II/III and glioblatoma (Abdel-Fattah et al., 2006). However, no associations with outcome or response to therapy have been reported, nor has a functional role of the HOX genes in glioma development been established.

These molecular signatures will be useful for patient stratification for specific therapies. The associations with outcome underline the need for development of multimodality treatments targeting not only the tumour cells, but including strategies aimed at the glioma stem-like cell compartment (identified by high HOX gene expression), and interfering with tumor host interaction that provides the specialized microenvironment relevant for the maintenance of tumour stem-like cells (the stem-cell niche). Better outcome was associated with gene clusters characterizing features of tumour host interaction including tumour vascularization and cell adhesion (G07, G14), and innate immune response. The positive association of high expression of tumour blood vessel markers (G07) with outcome may reflect improved perfusion of the active therapeutic drug. This signature may be of further interest for therapies aimed at targeting angiogenesis that are thought to improve blood perfusion of the tumors transiently.

Thus there is a profound need to develop an effective predictive method for identifying resistance to concomitant chemo-radiotherapy of a subject suffering from brain tumor and effective methods and compositions for treatment or prevention of this condition. The main problem is that to date, no efficient methods or strategies have been developed to overcome this problem and to identify patients benefiting from therapies.

### SUMMARY OF THE INVENTION

This object has been achieved by providing a method for predicting or diagnosing outcome of concomitant chemo-radiotherapy of a subject suffering from brain tumor comprising:

(a) obtaining a biological sample from said subject,
(b) measuring the expression of gene clusters associated with tumor resistance to the concomitant chemo-radiotherapy treatment wherein said gene clusters are selected from the group comprising HOX genes, G13 genes, G18 genes, G25 genes, G07 genes and G14 genes, biologically active fragment thereof and/or combinations thereof.
(c) comparing the expression level of said gene clusters to threshold values, wherein the high expression of HOX genes, G13 genes, G18 genes and G25 genes indicate high risk for brain tumor resistance to the concomitant chemo-radiotherapy treatment whereas the expression of G07 genes and G14 genes indicate better outcome to the concomitant chemo-radiotherapy treatment, and optionally evaluating the medical prognosis of said subject based on the comparison of step (c), and/or adapting the treatment of said subject.

A further object of the present invention is to provide a kit useful for predicting or diagnosing the tumor resistance in a subject treated with concomitant chemo-radiotherapy, said kit comprises a set of primers, probes or antibodies specific for one or more genes selected from gene clusters comprising HOX genes, G13 genes, G18 genes, G25 genes, G07 genes and G14 genes, biologically active fragment thereof and/or combinations thereof.

Another object of the invention is the use of modulators of expression of at least one gene selected from gene clusters comprising HOX genes, G13 genes, G18 genes, G25 genes, biologically active fragment thereof and/or combinations thereof in the preparation of a medicament for the treatment or prevention of tumor resistance in a subject suffering from a brain tumor, wherein said modulators are inhibitors.

Further object of the invention is the use of modulators of the biological activity of a protein encoded by at least one gene selected from gene clusters comprising HOX genes, G13 genes, G18 genes, G25 genes, biologically active fragment thereof and/or combinations thereof in the preparation of a medicament for treatment or prevention of tumor resistance in a subject suffering from a brain tumor, wherein said modulators are inhibitors or competitors.

### BRIEF DESCRIPTION OF THE FIGURES

FIG. 1. Sample Dendrogram S1(G1) and Gene Distance Matrix (G1) by Coupled Two-Way Clustering (CTWC).

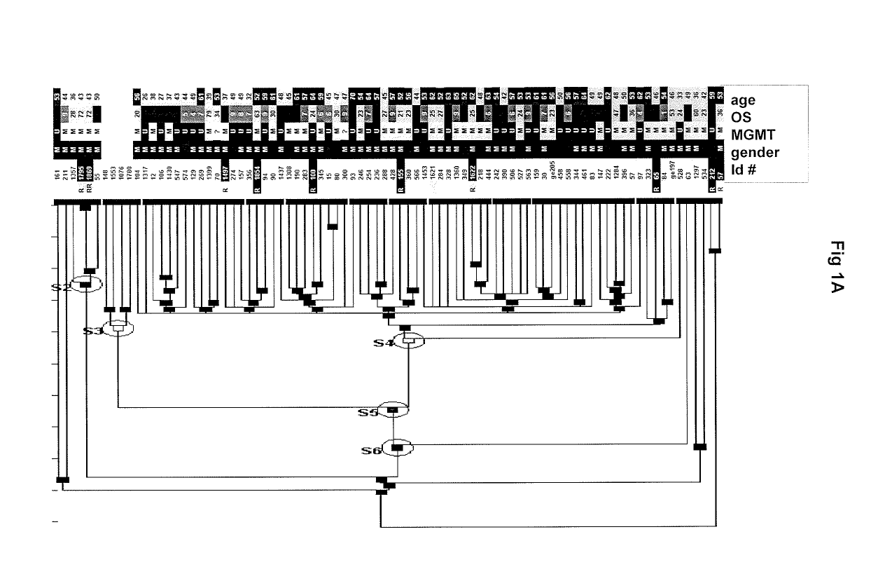

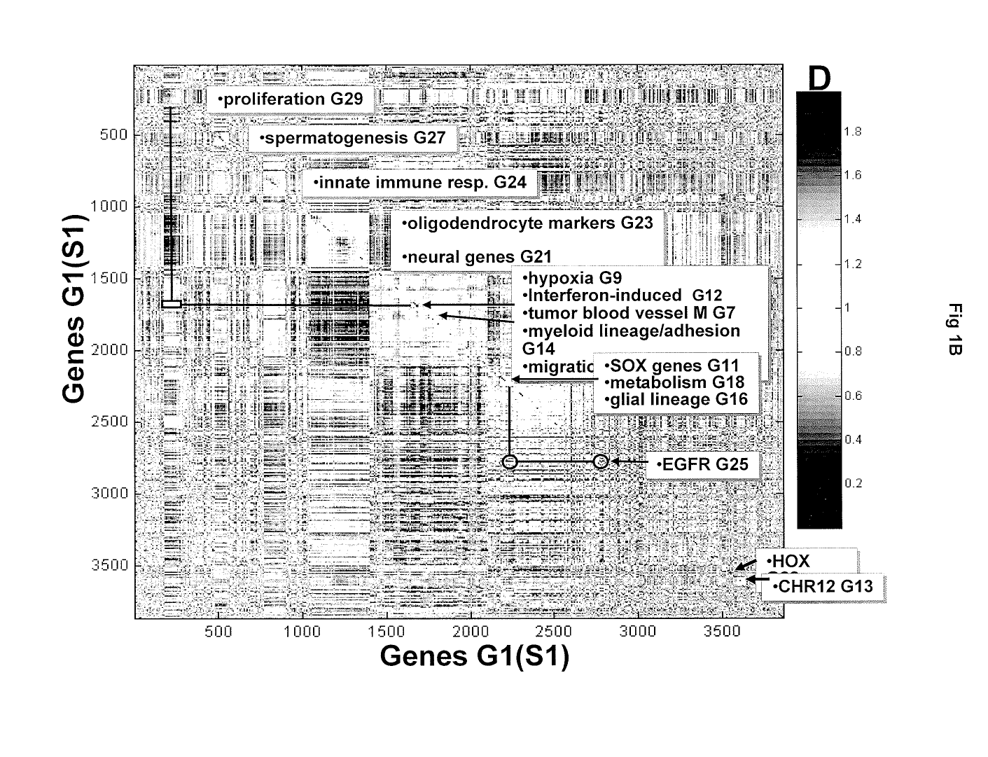

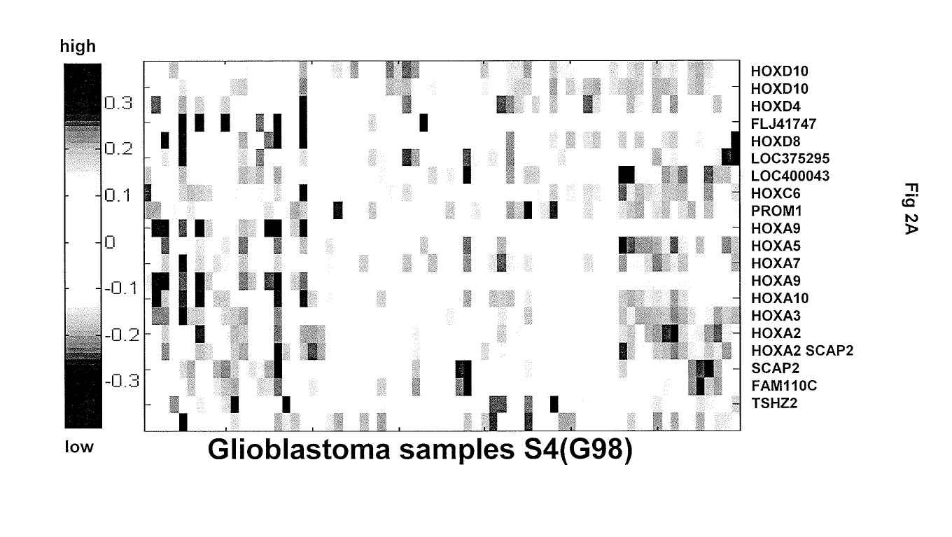

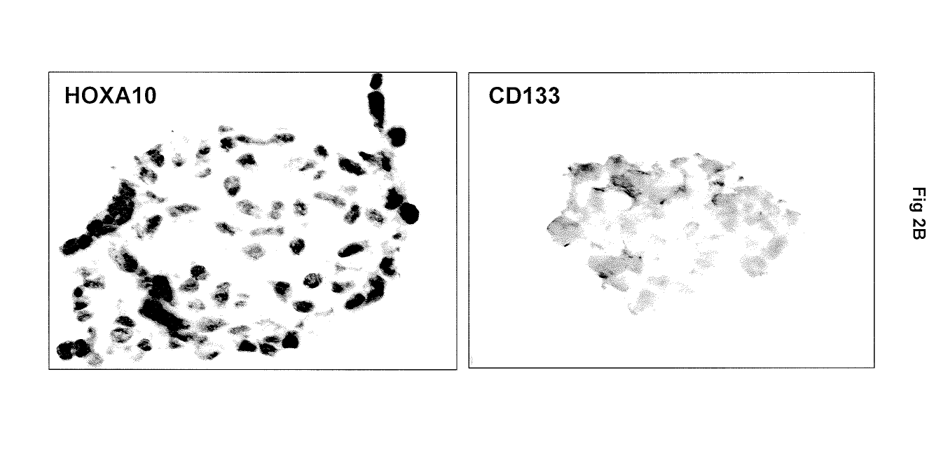

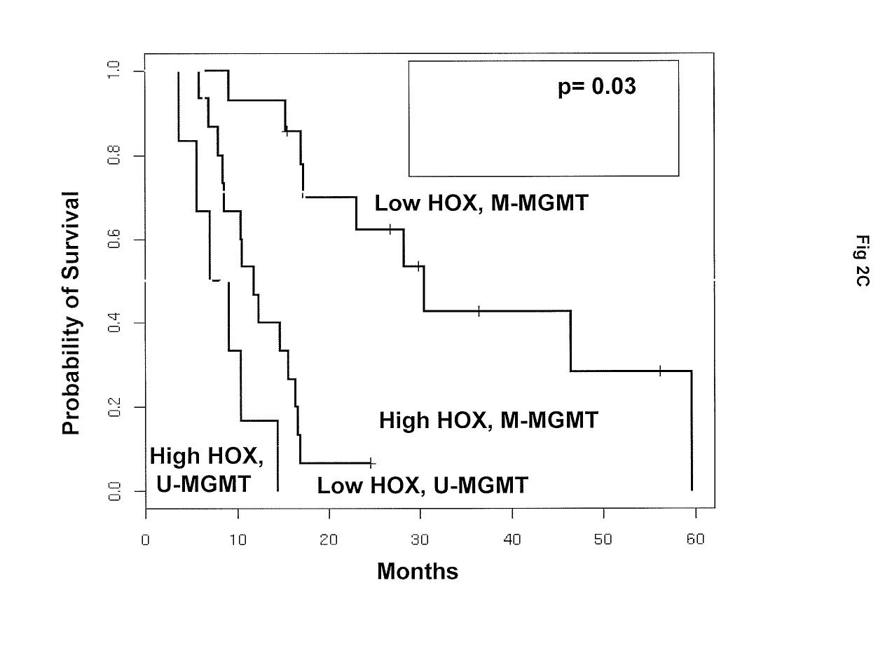

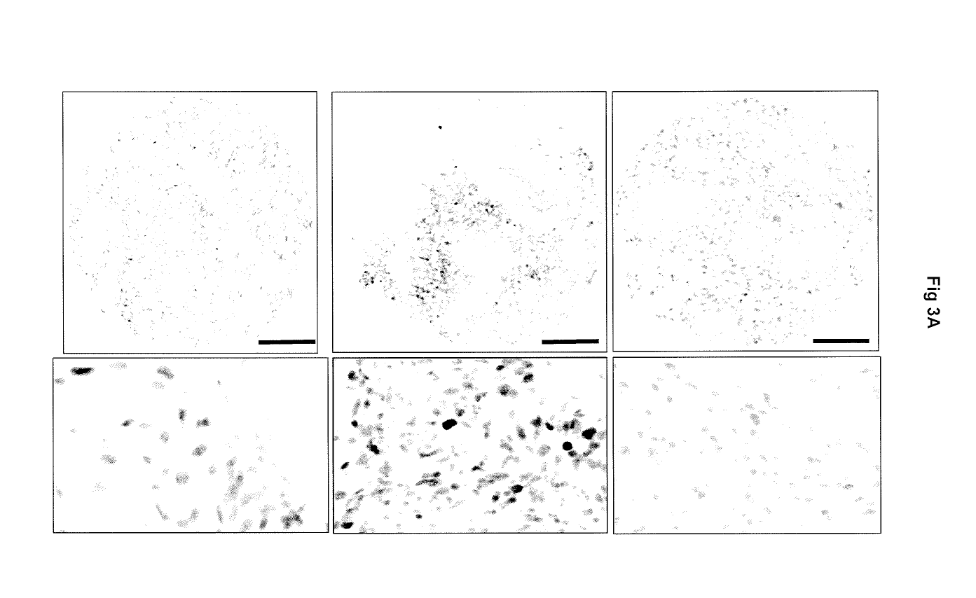

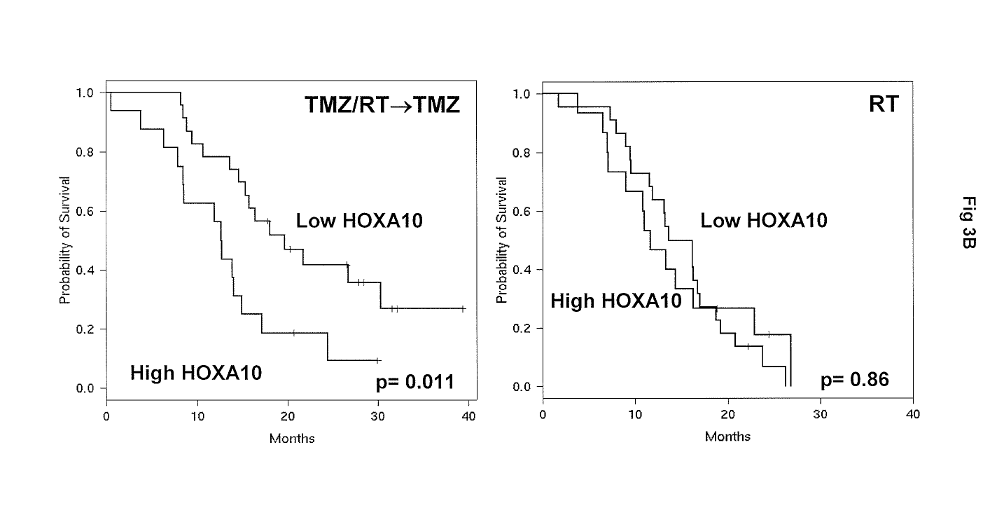

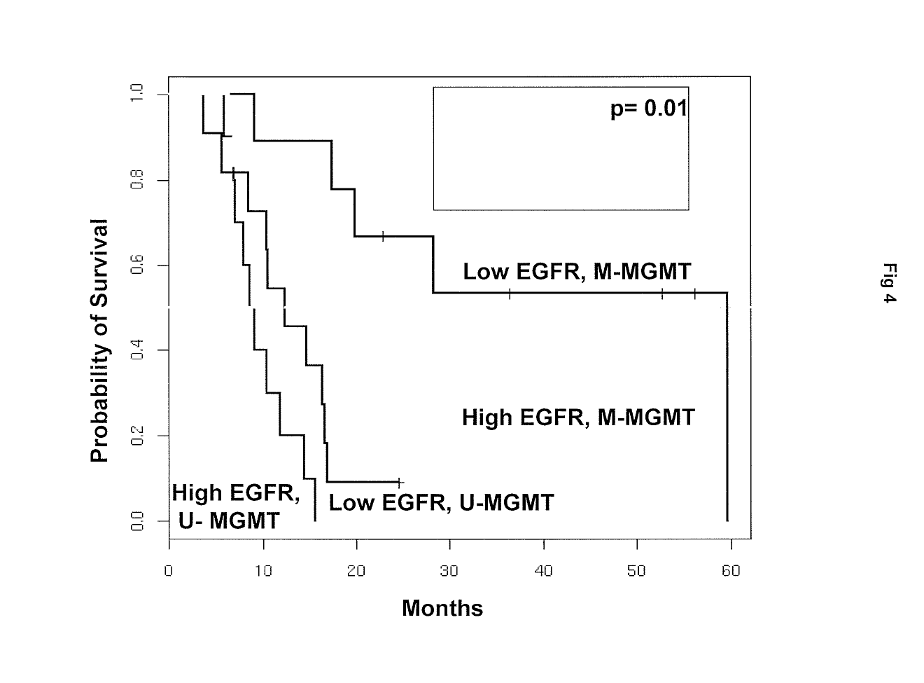

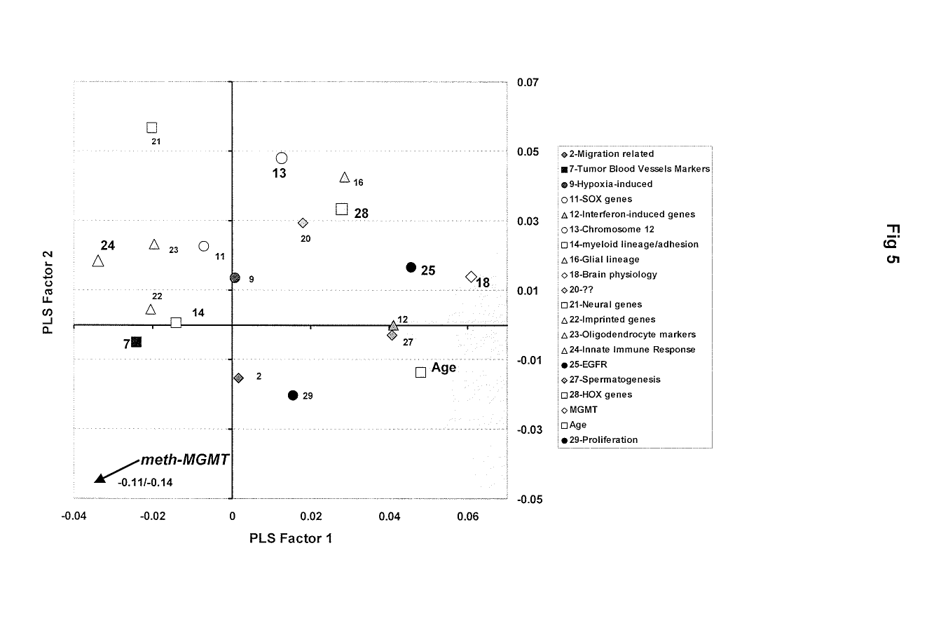

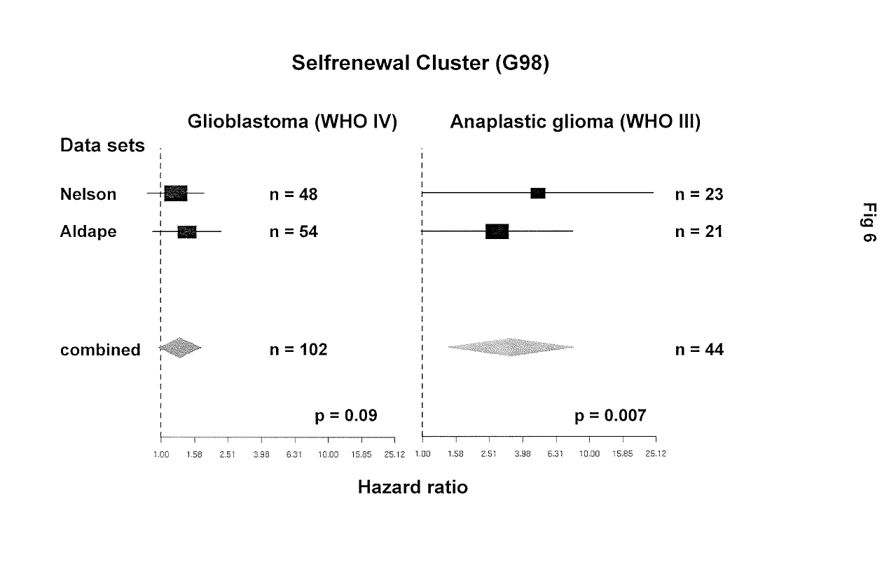

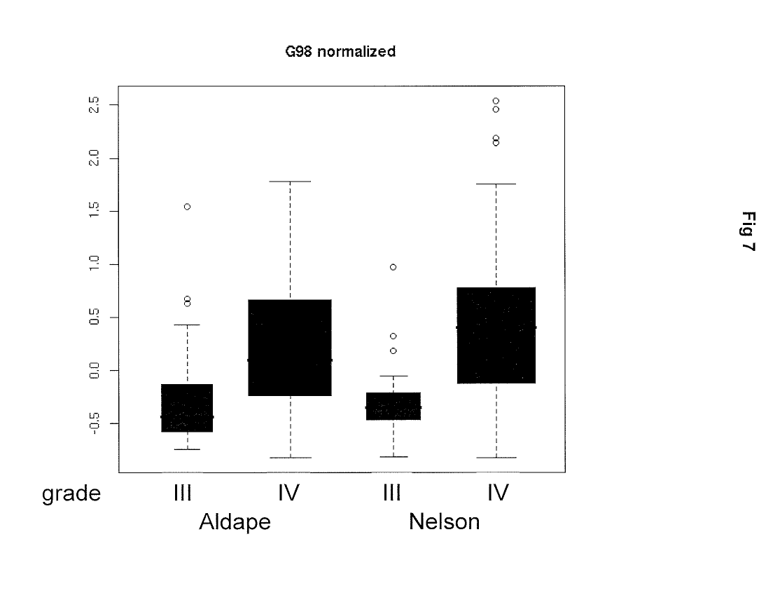

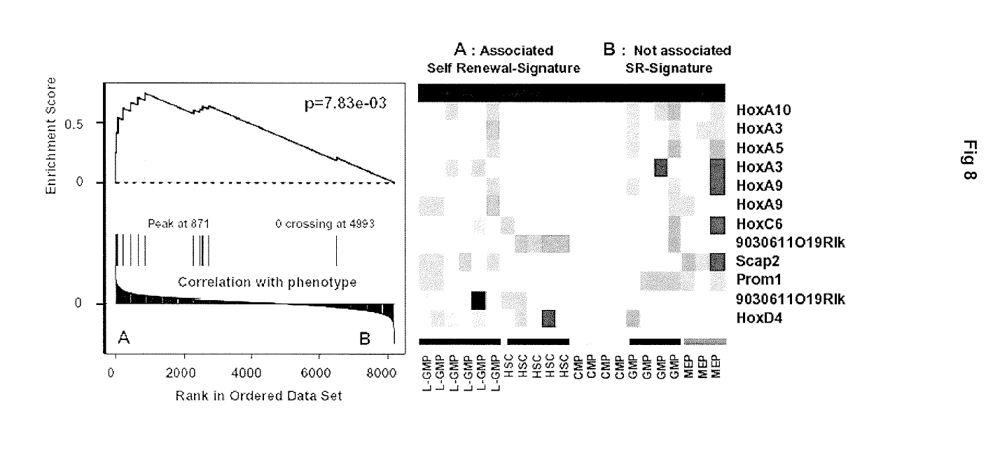

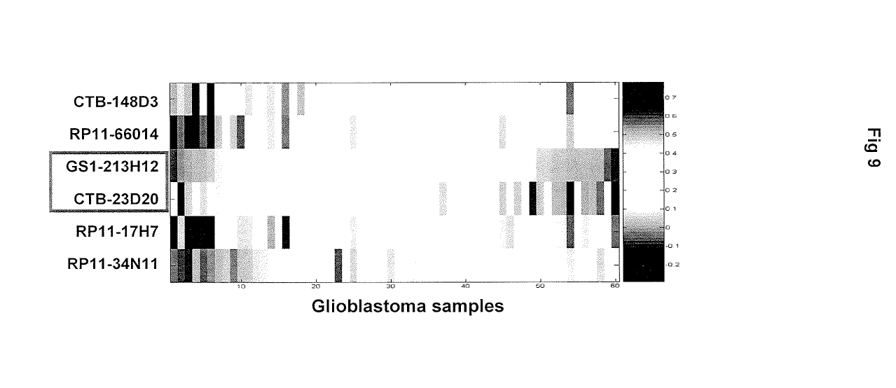

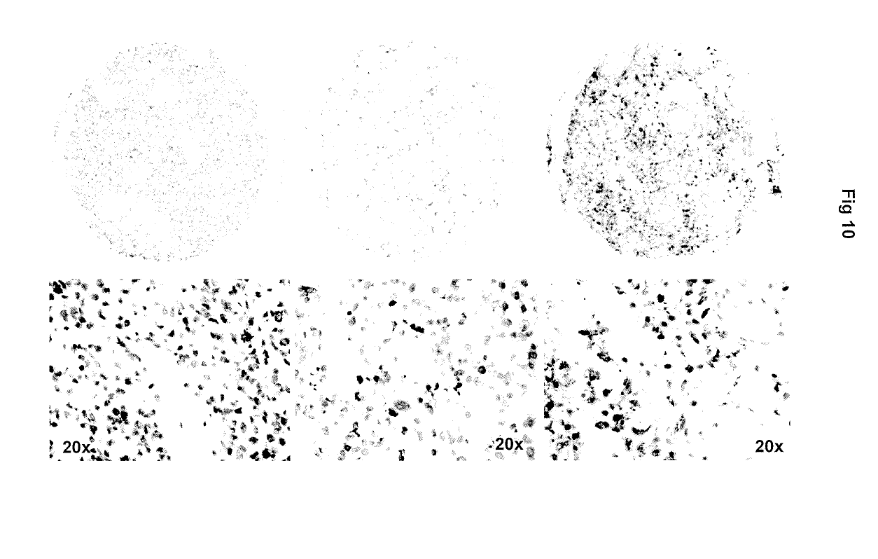

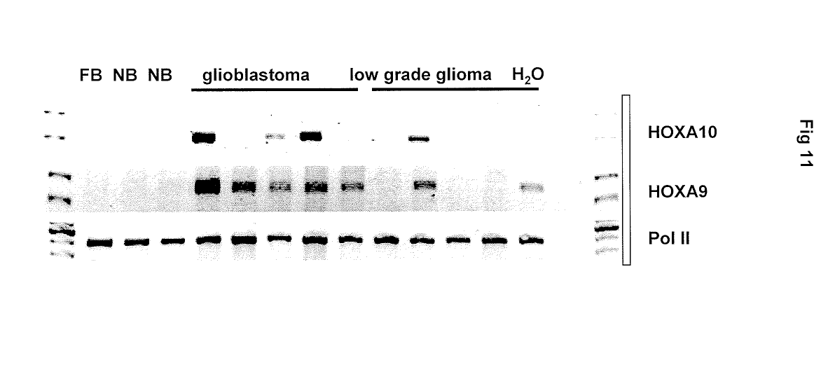

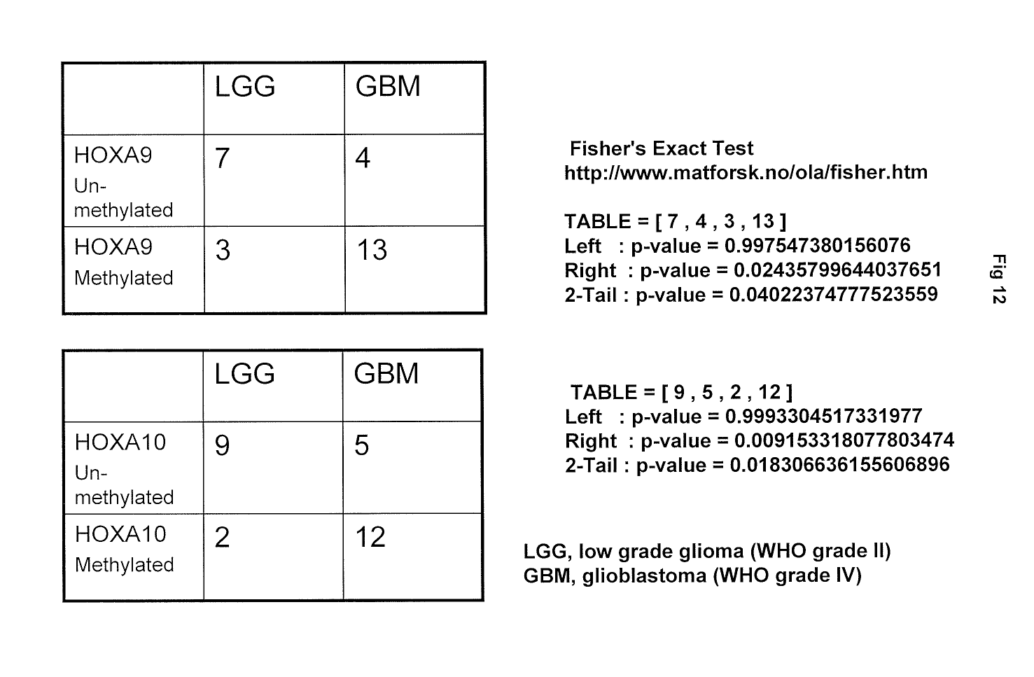

(A) Sample dendrogram S1(G1) emerging from clustering all 84 samples S1 with all genes G1. Stable sample clusters S2 to S6 emerge. Clinical information: age at diagnosis, green<50 years; brown>50 years; overall survival, OS, in months, green<9; red 9-18; pink>18; MGMT methylation status, grey, methylated; black, unmethylated; white unknown; gender, red, female; sample Id#, sample identification number, non tumoral brain tissue, yellow; recurrent glioblastoma, black; color code tumor pairs from same patient, black only recurrent glioblastoma. (see URL in Methods to view gene dendrogram and entire CTWC analysis)
(B): The distance matrix of all filtered genes (G1) (Euclidian distance) heatmap; blue, short distance (more similar), red, large distance. The “distance” ranges from 0 (corresponding to Pearson correlation=1, i.e. full similarity of the two expression profiles) to 2 (corresponding to anti-correlated profiles, Pearson=−1). Zero correlation, orange. Annotation is based on gene dendrogram G1(S1) (not shown). The distance matrix visualizes relationships between clusters as indicated by lines and arrows: e.g. G7 is in the center of a “supercluster”, reflecting close relationship with G9, G12, G14 and G2. In addition G7 is related to G29, but not G23 or G21.
(C): Respective distance matrices for two published data-sets with 48 and 54 glioblastoma (Freije et al., 2004; Phillips et al., 2006), comprising the common probe-sets (3649) and ordered according to the matrix in FIG. 1B. All 3 data-sets appear very similar, particularly visible for the largest clusters annotated in FIG. 1B.

FIG. 2. Relation of Glioblastoma Derived Hox Gene Signature with Survival

(A) Stable cluster G98 was found by clustering all genes G1 with samples clustered in S4 (69 glioblastoma), red, relative overexpression; blue relative underexpression.
(B) Glioblastoma-derived neurospheres (40×) cultured under stem cell conditions exhibit strong nuclear staining for HOXA10 and express CD133 as determined by immunohistochemistry. Representative neurospheres from two glioblastoma are shown.
(C) Kaplan-Meier survival estimates of the 42 patients treated with TMZ/RT→TMZ separated into high versus low expressors of G98 genes, High HOX, Low HOX (dichotomized according to CTWC sample dendrogram). The p-value of the log-rank test is shown for the two groups, stratified by the MGMT methylation status, M-MGMT, methylated MGMT; U-MGMT, unmethylated MGMT.

FIG. 3. HOXA10 Expression and Overall Survival in Independent Patient Cohort.

(A) HOXA10 expression determined by immunohistochemistry on TMA. Diameter of tissues in upper panel: 0.6 mm. First panel, high nuclear expression; second, focal high nuclear expression; third, no expression.
(B) Kaplan-Meier survival estimates of 39 patients randomized to TMZ/RT→TMZ and 37 patients randomized to RT, separated into high versus low expressors of HOXA10 as determined by immunohistochemistry on TMA.

FIG. 4. Association of EGFR Expression with Survival.

Kaplan-Meier curves of the 42 patients in the TMZ/RT→TMZ cohort divided between low and high expression of EGFR (probeset 201983_s_at, dichotomized according to a Gaussian mixture model). The p-value from log-rank test is shown for two groups defined by EGFR expression and stratified according to the MGMT methylation status. M-MGMT, methylated MGMT; U-MGMT, unmethylated MGMT.

FIG. 5. PLS model for survival Cluster X-weights were obtained by averaging the X-weights of their constituent genes. The clusters are numbered according to the legend and the description in Table 1. Age and MGMT methylation-status were used as covariates. Clusters of genes with X-weights that are nearest to the PLS factors, represented by the axes (PLS factor 1 and 2), and the farthest from the center of the plane contribute most to the PLS regression. The first two factors shown explain 66% of the survival outcome variations. Clusters of genes grouped in the upper and right side of the plane have a positive association with shorter survival (i.e. higher hazards), while those in the lower and left side are positively associated with longer survival (i.e. lower hazards).

FIG. 6. Correlation of G98 expression and survival in external datasets. High expression of the selfrenewal cluster G98 is significantly associated with worse outcome in two independent datasets comprising a total of 146 malignant glioma (P=0.007, hazard ratio: 1.46, 95% Confidence Interval: 1.11 to 1.92) (model adjusted for tumor grade WHO grade III and IV; age, and stratified for the dataset, Nelson; Aldape). The forest plots visualize the relationship between expression of G98 and outcome in the two datasets separated for tumor grade. The hazard ratio for GBM (WHO grade IV) is 1.29 (95% CI 0.97 to 1.72, P=0.09) (left panel). A hazard ratio of 3.35 (95% CI 1.39 to 8.08; P=0.007) is observed for the subset of anaplastic glioma (WHO grade III) (right panel)

FIG. 7. Box plot for expression of G98 in grade III and grade IV glioma of external datasets. G98 expression significantly differentiates anaplastic glioma (WHO grade III) from GBM (WHO grade IV). GBM exhibit significantly higher expression of G98 in both external data sets (Wilcoxon rank sum test with continuity correction, Nelson, P<0.001; Aldape, P=0.002).

FIG. 8. HOX-signature and Self-renewal. Genes in the HOX gene cluster G 98 are significantly (P=0.008) enriched in the mouse expression data among genes differentiating mouse hematopoetic cell populations into two classes, A) self renewal-assocaited samples: granulocyte macrophage progenitor-derived leukemic cells (L-GMP), and hematopoietic stem cells (HSC); and B) non self renewal associated samples: common myeloid progenitors (CMP), granulocyte macrophage progenitors (GMP), and megakaryocyte erythrocyte progenitors (MEP). Heat map: dark grey=up regulated; light grey=down regulated, in comparison to the median expression value.

FIG. 9. Array-CGH data of the HOX gene region. aCGH data for the chromosomal region around the HOX gene cluster on chromosome 7. Every row is a marker, ordered by chromosomal location. Every column is a sample (n=60), ordered by the copy number of BAC GS1-213H12 (bacterial artificial chromosome) using SPIN software. The HOXA gene cluster resides between markers GS1-213H12 and CTB-23D₂O that appear to be amplified more frequently and more strongly than the neighboring markers.

FIG. 10. Expression of CD163 in GBM Immunohistochemistry for CD163 (Novocastra; NCL-163; dilution 1:800) on representative paraffin-embedded GBM. Diameter of tissues in upper panel is 0.6 mm. CD163 is a marker for M2-polarized macrophages.

FIG. 11. Expression of HOXA10 & 9 RNA in Glioma. FB, fetal brain; NB, normal brain

FIG. 12. Promoter Methylation of HOXA9 & 10 increases with Tumor Malignancy in Glioma.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Unless defined otherwise, all technical and scientific terms used herein have the same meaning as is commonly understood by one of skill in art to which the subject matter herein belongs.

As used herein, the following definitions are supplied in order to facilitate the understanding of the present invention.

The term “comprise” is generally used in the sense of include, that is to say permitting the presence of one or more features or components.

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

As used herein, the terms “peptide”, “protein”, “polypeptide”, “polypeptidic” and “peptidic” are used interchangeably to designate a series of amino acid residues connected to the other by peptide bonds between the alpha-amino and carboxy groups of adjacent residues.

Glioblastoma are notorious for resistance to therapy which has been attributed to DNA repair proficiency, a multitude of deregulated molecular pathways, and more recently to the particular biological behavior of tumor stem-like cells. In the present invention the Applicants identified molecular profiles specific for treatment resistance to the current standard of care of concomitant chemo-radiotherapy with the alkylating agent temozolomide.

Recent concepts for cancer development suggest that a minority population of cancer stem-like cells may determine the biological behavior of tumors, including response to therapy. Failure to cure cancer has been attributed to the fact that therapies are aimed at the tumor bulk without significantly harming tumor stem-like cells (Reya et al., 2001), supported by experimental evidence in a respective mouse model showing that this glioblastoma subpopulation of cells is more resistant to radiotherapy (Bao et al., 2006). Facilitated by markers differentiating stem-cells and progenitors of the different lineages, the origin of leukemic stem-cells has been traced back to hematopoietic stem-cells as well as progenitor populations that have acquired “self-renewal” properties (Krivtsov et al., 2006). In contrast, the origin and concept of glioma stem-like cells remains to be fully elucidated. CD133⁺ has been postulated to be a glioma stem-cell marker, as this subpopulation of glioma-derived cells seems to have a higher potential to generate and maintain tumors in vivo (Bao et al., 2006).

The Applicants have identified several biological processes associated with resistance or responsiveness to combined chemo-radiotherapy that provide important information guiding novel treatment strategies and aiming at individualized therapy. Intriguingly, an expression signature associated with resistance shows high similarity with a stem cell-related “self-renewal” signature (Krivtsov et al., 2006).

The Applicants provide first clinical evidence for the implication of a “glioma stem-cell” or “self-renewal” phenotype in treatment resistance of glioblastoma. Biological mechanisms identified here to be relevant for resistance will guide future targeted therapies, and respective marker development for individualized treatment and patient selection.

Gene expression signatures relevant for treatment resistance to TMZ/RT→TMZ have been identified in a prospectively treated population of glioblastoma patients. While epigenetic inactivation of the MGMT gene promoter remained the most prominent predictive factor, expression signatures allowed identification of patient sub-groups who may benefit from specific additional therapies targeting particular mechanisms of resistance.

As an independent predictive factor of resistance the Applicants have identified a HOX-dominated gene cluster, evocative of a “self-renewal signature”. Strong HOXA10 expression of glioblastoma derived neurospheres is in line with a role of HOX-genes in the glioma stem-like cell compartment. These findings provide the first clinical evidence for the relevance of a stem-like cell phenotype in treatment resistance of glioblastoma.

A “gene cluster” refers to a set of two or more genes that serve to encode for the same or similar products.

In leukemia expression of translocation-related fusion proteins lead to MLL-mediated chromatin remodeling associated with re-expression of HOX-genes (Dorrance et al., 2006). In glioblastoma, however, such fusion proteins have not been described, and no indications from the present data-set link MLL-expression with the “self-renewal signature”. The Applicant's provide evidence that the HOX-dominated gene signature emerges with malignant progression to glioblastoma, and may be acquired in some glioblastoma by low level amplification, the latter supporting the notion that gliomas may also arise from progenitors, in agreement with mouse models (Bachoo et al., 2002). The identification of GADD45G as part of the HOX-signature may provide further evidence for an enhanced DNA repair potential that recently has been associated with radiation resistance of glioma stem cells (Bao et al., 2006).

These molecular and clinical data underscore the importance of the self-renewal phenotype which could be explored as a potential treatment target (Stupp and Hegi, 2007). First efforts blunting glioma stem-cell-related self-renewal properties of tumors suggest that strategies forcing differentiation, e.g. mediated by cytokines such as BMP4, may be promising (Piccirillo et al., 2006).

Targeting resistance to TMZ/RT→TMZ associated with overexpression of the EGFR gene is of particular clinical interest, since this alteration affects a large proportion of patients. CDK4 and MDM2 genes, both proto-oncogenes showed amplification-mediated overexpression, are identified in the present invention as associated with worse outcome.

Surprisingly, good prognosis was associated with increased expression of a signature for tumor endothelium markers. This signature may predict improved cytotoxic activity by means of better perfusion of the tumor with the chemotherapy agent TMZ. This is in accordance with the concept suggesting that anti-angiogenic agents may temporarily lead to “normalization” of aberrant tumor vasculature resulting in more efficient delivery of drugs and oxygen to the tumor (Batchelor et al., 2007). In a recent trial addition of the anti-angiogenic integrin-inhibitor cilengitide appears to confer increased anti-tumor activity in conjunction with TMZ/RT→TMZ in patients with a methylated MGMT gene promoter (Stupp et al., 2007).

Another interesting insight of the present invention suggests infiltration of M2-polarized macrophages into the tumors. The altered capacity of these glioma-infiltrating macrophages to induce effective anti-tumor T-cell response may obstruct therapeutic strategies aimed at boosting adaptive immunity against the tumor. M2-polarization is driven by tumor-derived and T-cell-derived cytokines (Mantovani et al., 2002), consistent with the well-known expression of the immunosuppressive cytokines TGF-beta and Interleukin 10 in malignant glioma (Kjellman et al., 2000). Thus, for effective immunotherapy/vaccination full resection of the tumor may be required to remove the microenvironment conferring immunosuppression and tolerance.

The gene signatures identified in the present invention is associated with outcome underline the need for development of multimodality treatments targeting not only the tumor cells, but including strategies aimed at the glioma stem-like cell compartment, and interfering with tumor host interaction that provides the specialized microenvironment relevant for the maintenance of tumor stem-like cells (the stem-cell niche), angiogenesis, and immune response. The present invention is useful to guide a rational choice of agents, targets, trial design, and appropriate patient selection, incorporating biomarkers defining mechanisms of response and resistance.

Epigentic silencing of HOX-genes by promoter methylation increases during malignant progression of glioma. Prognostic marker in the tissue and body-fluids such as cerebrospinal fluid (CSF) and blood.

Progression of gliomas to glioblastoma (WHO grade IV) is associated with increasing expression of HOX genes (eg HOX genes in cluster G98/G28).

Investigation of HOXA10 and HOXA9 in glioma revealed epigenetic promoter deregulation associated with tumor malignancy, reflected by the presence of respective hypermethylated gene promoter alleles in most GBM (12/17 and 13/17, respectively) in contrast to lower grade gliomas (2/11 and 3/10; Fisher exact test p=0.009 and p=0.02, FIG. 11). In all tumors unmethylated alleles were also present. Tumors with methylated promoters showed HOXA10 and HOXA9 expression, while, normal brain showed no methylation and no expression, like most low grade gliomas (FIG. 12).

The high frequency of HOXA10 and 9 promoter methylation in glioblastoma makes these very good prognostic markers in the tissue but also for detection in cerebrospinal fluid (CSF) or blood. In the CSF and blood they have not only a prognostic value on their own, prediction of high grade glioma, but also may serve as marker for tumor derived DNA. The promoter methylation can be detected by many different technologies including MSP, pyrosequencing, MS etc.

Thus, the present invention relates to a method for predicting or diagnosing outcome of concomitant chemo-radiotherapy of a subject suffering from brain tumor comprising:

(a) obtaining a biological sample from said subject,
(b) measuring the expression of gene clusters associated with tumor resistance to the concomitant chemo-radiotherapy treatment wherein said gene clusters are selected from the group comprising HOX genes, G13 genes, G18 genes, G25 genes, G07 genes and G14 genes, biologically active fragment thereof and/or combinations thereof.
(c) comparing the expression level of said gene clusters to threshold value, wherein the high expression of HOX genes, G13 genes, G18 genes and G25 genes indicate high risk for brain tumor resistance to the concomitant chemo-radiotherapy treatment whereas the expression of G07 genes and G14 genes indicate better outcome to the concomitant chemo-radiotherapy treatment, and optionally evaluating the medical prognosis of said subject based on the comparison of step (c), and/or adapting the treatment of said subject.

The term “radiotherapy” refers to the use of ionizing radiation as part of cancer treatment to control malignant cells. It is also common to combine radiotherapy with surgery, chemotherapy, hormone therapy or combinations thereof. Most common cancer types can be treated with radiotherapy in some way. The precise treatment intent (curative, adjuvant, neoadjuvant, or palliative) will depend on the tumor type, location, and stage, as well as the general health of the patient.

The term “chemotherapy” generally refers to a treatment of a cancer using specific chemotherapeutic/chemical agents. A chemotherapeutic agent refers to a pharmaceutical agent generally used for treating cancer. The chemotherapeutic agents for treating cancer include, for example, cisplatin, carboplatin, etoposide, vincristine, cyclophosphamide, doxorubicin, ifosfamide, paclitaxel, gemcitabine, docetaxel, and irinotecan and platinum-based anti-cancer agents, including cisplatin and carboplatin. Other chemotherapy classes comprise tyrosine kinase inhibitors such as gefitinib, imatinib; farnesyl transferase inhibitors including lonafarnib; inhibitors of mammalian targets of rapamycin (mTOR) such as evereolimus; angiogenesis inhibitors including bevacizumab, sunitibid and cilengitide; inhibitors of PKC; PI3K and AKT. More specifically, the chemotherapeutic agents of the present invention include alkylating agents such as Temozolomide or carmustine.

The term “concomitant chemo-radiotherapy” is used when these two treatments (chemotherapy and radiotherapy) are given either at the same time, or almost at the same time, for instance one after the other, or on the same day, etc.

According to the present invention, the preferred agent for chemotherapy is temozolomide (TMZ).

The term “adapting the treatment” generally refers to the choice of a treatment among different options, based on the specificities of the disease, concomitant pathologies or patient conditions, or the switch from one treatment to another in the course of the therapy because of the non-response, progression or resistance of the disease to the initial treatment, with the intent to offer to the patients the beast treatment for his diseases under the given circumstances.

The biological sample, used in the method of the invention, is a biopsy of brain tumor. Preferably, the biological sample is a glioblastoma sample.

In the method of the present invention, the subject is a mammal and preferably a human.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment. However, in other embodiments, the subject can be a normal subject.

In the method of the present invention the HOX gene cluster includes one or more genes selected from the group comprising GADD45G, SCAP2, HOXD4, HOXC6, HOXA9, HOXA10, HOXA5, HOXA2, SCAP2, LOC400043, LOC375295, HOXD10, HOXD8, HOXA3, HOXA7, HOXD10, FLJ41747, PROM1, TSHZ2, and FAM110C. Preferably HOX genes are HOXA9 and HOXA10.

In the method of the present invention the G13 gene cluster includes one or more genes selected from the group comprising B4GALNT1, AVIL, OS9, CDH1, CDK4, TSPAN31, METTL1, MDM2, CYP27B1, CPM, TSFM, FAM119B, SLC26A10, NUP107, KIAA1267, MGC5370, MARCH9, XRCC6BP1, DTX3, and RGS8. Preferably G13 genes are CDK4 and MDM2 (see table 11).

The expression level of said gene clusters is then compared to threshold value, said “threshold value” refers to, e.g. the expression level of all RNA transcripts or their expression products in said sample or of a reference set of RNA transcripts or their expression products in said sample. Usually, a high expression of HOX genes, G13 genes, G18 genes and G25 genes indicate high risk for brain tumor resistance to the concomitant chemo-radiotherapy treatment whereas the expression of G07 genes and G14 genes indicate better outcome to the concomitant chemo-radiotherapy treatment, and optionally evaluating the medical prognosis of said subject based on the comparison of expression level of said gene clusters, and/or adapting the treatment of said subject.

The term “prognosis” is recognized in the art and encompasses predictions about the likely course of disease or disease progression, particularly with respect to likelihood of disease remission, disease relapse, tumor recurrence, metastasis, and death.

In the method of the present invention the G18 gene cluster includes one or more genes selected from the group comprising SLC1A3, ITPKB, MAOB, F3, BBOX1, DTNA, NDP, NR2E1, P2RY1, CA2, SLC7A11, AQP4, MLC1, CENTD1, SLC25A18, ITGB8, PAX6 and FLJ25530. Preferably G18 genes are AQP1 and AQP4.

In the method of the present invention the G25 gene cluster includes one or more genes selected from the group comprising EGFR, SOCS2, SEC61G, EYA2, SHOX2, EMILIN3, MASP1, FOXO1A, LHFP, and PDZD2. Preferably G25 gene is EGFR.

In the method of the present invention the G07 gene cluster includes one or more genes selected from the group comprising COL1A1, KDELR2, LAMC1, COL6A3, LAMB1, LUM, COL3A1, NID1, VWF, LAMA4, MGP, SEC24D, COL1A2, PCOLCE, FMOD, FBN1, CD93, ADAM12, LOXL2, COL5A1, IGFBP6, KDELR3, TPM2, NID2, EDNRA, CDH5, LTBP2, ENPEP, SRPX2, ANGPT2, SERPINH1, PDLIM1, COL6A2, MXRA5, FN1, ANGPT2, COL13A1, FN1, COL4A2, COL4A1, NRP1, MYO1B, OLFML2B, SNAI2, PLXDC1, LXN, ELTD1 NOX4, COL5A2, ETS1, CTHRC1, MGC4677///L00541471, PELO, FAM20A, LOC493869, and GJA7.

In the method of the present invention the G14 gene cluster includes one or more genes selected from the group comprising ALDH1A3, MME, THBS4, BMP5, MEOX1, COMP, GAS2, SEPT6///N-PAC, SOSTDC1, OLFML1, RP6-213H19.1, CYTL1, PRR16, TNMD, FNDC1, GLT8D2, CDC42EP5, SCARA5, COL12A1, DNM3, HMCN1 and MKX. Preferably G14 genes are PRR16, MEOX1, MKX and BMP5.

Comparing the expression level of said gene clusters to threshold value, wherein the high expression of HOX genes, G13 genes, G18 genes and G25 genes indicate high risk for brain tumor resistance to the concomitant chemo-radiotherapy treatment whereas the expression of G07 genes and G14 genes indicate better outcome to the concomitant chemo-radiotherapy treatment, and optionally evaluating the medical prognosis of said subject based on the comparison of step (c), and/or adapting the treatment of said subject.

The Cluster Indexes (CI) are defined as follows (the “Clusters”, Gxx: gene cluster G07, G13, G14, G18, G25, HOX genes, as defined in the Tables):

GxxCI=log₂ [(CTE₁+Gxx Cluster Metagene Score)/(CTE₂+Reference Metagene Score)]+CTE₃

The Cluster metagene score is a weighted average of the expression values of the genes in the Cluster (see, respective Tables of clusters) measured within the tumor biopsy.

$$Gxx Metagene Score = \frac{1}{n} {\sum }_{i = 1}^{n} ( {Gxx\_Gene}_{i} \cdot {ks}_{i} )$$

wherein
n=2 to number of genes in the Cluster
“GxxCuster Gene,” represents the expression value of each gene in the cluster.
ksᵢ defines the importance of the corresponding gene in the calculation of the weighted average of the Cluster Metagene Score. The variable ksᵢ may take any positive real value within the range of zero (inclusive) and 1000 times the maximal expression value of the gene in the Cluster included in the calculation of the Cluster Metagene score.

Preferably the expression value of more than 2 genes in the Cluster are used to calculate the Cluster Metagene Score.

The purpose of the variable ksᵢ is to adjust (or correct) for the difference in expression magnitude between genes in the Cluster and therefore will make these expression values more similar to all other genes in the Cluster included in the calculation of the Cluster Metagene Score.

The variable CTE1 may take any real value within the range of plus/minus 1000 times the average of the Cluster Metagene Score. The purpose of the CTE1 variable is to adjust for differences in efficiency in extracting the mRNA of genes in the Cluster from the tumor sample relative to the reference genes.

The reference metagene score is a weighted average of the expression values of the reference genes (see Table of reference genes) measured within the tumor biopsy.

$$The Reference Metagene Score == \frac{1}{n} {\sum }_{t = 1}^{n} ( {Reference\_Gene}_{t} \cdot {kr}_{t} )$$

wherein
n=2 to 6
“Reference Geneₜ” represents the expression value of each reference gene.

krₜ defines the importance of the corresponding reference gene in the calculation of the weighted average of the Reference Metagene Score. The variable krₜ may take any positive real value within the range of zero (inclusive) and 1000 times the maximal expression value of the reference gene included in the calculation the Reference Metagene Score.

The purpose of the variable krₜ is to adjust (or correct) for the difference in expression magnitude between reference genes and therefore will make these expression values more similar to all other reference genes included in the calculation of the Reference Metagene Score.

The variable CTE2 may take any real value within the range of plus/minus 1000 times the average of the reference metagene score. The purpose of the CTE2 variable is to adjust for differences in efficiency in extracting the mRNA of reference genes from the tumor sample relative to the genes in the Cluster.

The purpose of the variable CT3 is to adjust for systematic bias due to experimental measurements.

A tumor sample is considered as being high expressor if the score CI is greater than the threshold TH1 (i.e. CI>TH1), which is indicative of resistance to chemoradiotherapy true for HOX genes, G13, G18 and G25; but of sensitivity to chemoradiotherapy true for clusters G7 and G14.

A tumor sample is considered as being low expressor if the score CI is lower than the threshold TH2 (i.e. CI<TH2), which is indicative of sensitivity to chemoradiotherapy true for HOX genes, G13, G18 and G25; but of resistance to chemoradiotherapy true for clusters G7 and G14.

The variables TH1 and TH2 can take any real value between −50 and +50.

The purpose TH1 constant is to adjust for the desired sensitivity and specificity in declaring a tumor sample as having a high Cluster Score. As the threshold TH1 increases, there will be an increase in the true positive rate when classifying a tumour sample as being high expressor.

The purpose of the TH2 constant is to adjust for the desired sensitivity and specificity in declaring a tumour sample as being low expressor. As the value of TH2 decreases, the higher will be the true positive rate of classifying a sample as being low expressor.

The use of both constant brings the advantage of controlling specificity and selectivity of samples being low and high expressor and thus leaving a security margin for samples having “dubious” (i.e. ambiguous) values.

According to the method of the present invention, the measuring of the expression of genes associated with tumor resistance to concomitant chemo-radiotherapy treatment is obtained by a method selected from the group consisting of:

(a) detecting RNA levels of said gene, and/or
(b) detecting a protein encoded by said gene, and/or
(c) detecting a biological activity of a protein encoded by said gene.

The detecting of RNA levels is obtained through Microarray hybridization, real-time polymerase chain reaction, Northern blot, In Situ Hybridization, sequencing-based methods, quantitative reverse transcription polymerase-chain reaction or RNAse protection assay.

The detecting of protein levels is obtained through Western blot, immunoprecipitation, immunohistochemistry, ELISA, Radio Immuno Assay, proteomics methods, or quantitative immunostaining methods.

The present invention further relates to a method for predicting or diagnosing the brain tumor in a subject, comprising:
(a) obtaining a biological sample from said subject,
(b) analyzing the epigenetic changes such as promoter methylation of HOXA10 and HOXA9 gene in glioblastoma, wherein the high frequency of HOXA10 and HOXA9 promoter methylation in glioblastoma is associated with tumor malignancy.

The said biological sample is body fluid, preferably cerebrospinal fluid (CSF) or blood.

The present invention also relates to a method for treatment or prevention of tumor resistance in a subject suffering from a brain tumor. The present invention encompasses the use of modulators of expression of at least one gene selected from gene clusters comprising HOX genes, G13 genes, G18 genes, and G25 genes, biologically active fragment thereof and/or combinations thereof in the preparation of a medicament for the treatment or prevention of tumor resistance in a subject suffering from a brain tumor.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder.

“Prevention” as used herein means that the administration of the modulator(s) as described results in a reduction in the likelihood that a subject at high risk for tumor resistance, relapse and/or metastatic progression after targeted anti-tumor therapy, radiotherapy, chemotherapy, or combination thereof will indeed develop said tumor resistance, relapse and/or metastatic progression. Preferably, in the context of the present invention, this phrase means that the administration of the modulator(s) results in the reduction of the likelihood or probability that a subject at risk for developing insulin-dependent diabetes will indeed develop tumor resistance, relapse and/or metastatic progression.

“Biologically active” means affecting any physical or biochemical properties of a living organism or biological process. Biologically Active Substance refers to any molecule or mixture or complex of molecules that exerts a biological effect in vitro and/or in vivo, including pharmaceuticals, drugs, proteins, peptides, polypeptides, hormones, vitamins, steroids, polyanions, nucleosides, nucleotides, nucleic acids (e.g. DNA or RNA), nucleotides, polynucleotides, etc.

“Fragments”, as referred to genes, are sequences sharing at least 40% nucleotides in length with the respective sequence of the gene. These sequences can be used as long as they exhibit the same biological properties as the native sequence from which they derive. Preferably these sequences share more than 70%, preferably more than 80%, in particular more than 90% nucleotides in length with the respective sequence from which it derives.

These fragments can be prepared by a variety of methods and techniques known in the art such as for example chemical synthesis.

In the present invention, said modulators of expression of at least one gene selected from gene clusters comprising HOX genes, G13 genes, G18 genes, and G25 genes, biologically active fragment thereof and/or combinations thereof are preferably inhibitors, which comprise RNA antisense, said RNA antisense comprising a nucleotide sequence complementary to a coding sequence of at least one gene selected from gene clusters comprising HOX genes, G13 genes, G18 genes, G25 genes, biologically active fragment thereof and/or combinations thereof.

According to the present invention, the inhibitors of expression of at least one gene selected from gene clusters comprising HOX genes, G13 genes, G18 genes, and G25 genes, biologically active fragment thereof and/or combinations thereof are also RNA interferents.

Furthermore, the inhibitors of expression of at least one gene selected from gene clusters comprising HOX genes, G13 genes, G18 genes, and G25 genes, biologically active fragment thereof and/or combinations thereof comprise an antibody, or an immunologically active fragment thereof, that binds to a protein encoded by any gene selected from gene clusters comprising HOX genes, G13 genes, G18 genes, G25 genes, biologically active fragment thereof and/or combinations thereof.

The present invention also relates to the use of modulators of the biological activity of a protein encoded by at least one gene selected from gene clusters comprising HOX genes, G13 genes, G18 genes, G25 genes, biologically active fragment thereof and/or combinations thereof in the preparation of a medicament for treatment or prevention of tumor resistance in a subject suffering from a brain tumor.

In the present invention, said modulators of the biological activity of a protein encoded by at least one gene selected from gene clusters comprising HOX genes, G13 genes, G18 genes, G25 genes, biologically active fragment thereof and/or combinations thereof are preferably inhibitors or competitors.

The inhibitor of the biological activity of a protein encoded by at least one gene selected from gene clusters comprising HOX genes, G13 genes, G18 genes, G25 genes, biologically active fragment thereof and/or combinations thereof is an antibody or immunologically fragment thereof that binds to a protein encoded by at least one gene selected from gene clusters comprising HOX genes, G13 genes, G18 genes, G25 genes, biologically active fragment thereof and/or combinations thereof.

The competitors are compounds able to disturb interaction between a protein and a receptor thereof, said protein being encoded by at least one gene selected from gene clusters comprising HOX genes, G13 genes, G18 genes, G25 genes, biologically active fragment thereof and/or combinations thereof.

In the context of the present invention, the RNA antisense reduces the expression of the specific target gene. For example, the RNA antisense can contain one or more nucleotides which are complementary to one or more gene sequences selected from gene clusters comprising HOX genes, G13 genes, G18 genes, G25 genes, biologically active fragment thereof and/or combinations thereof.

The antisense nucleic acids (DNA or RNA) of the present invention act on cells producing the proteins encoded by genes associated with tumor resistance to the concomitant chemo-radiotherapy, by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid (DNA or RNA) of the present invention can be made into an external preparation, including a liniment or a poultice, by admixing it with a suitable base material which is inactive against the nucleic acid.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids of the present invention can be given to the patient by direct application onto the ailing site or by injection into a blood vessel so that it will reach the site of ailment. An antisense-mounting medium can also be used to increase durability and membrane-permeability. Examples include, but are not limited to, liposomes, poly-L-lysine, lipids, cholesterol, lipofectin or derivatives of these. The dosage of the inhibitory nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein encoded by one or more genes selected from the group comprising HOX genes, G13 genes, G18 genes, G25 genes, biologically active fragment thereof and/or combinations thereof and are thereby useful for suppressing the biological activity of said protein. In addition, expression-inhibitors, comprising antisense nucleic acids of the present invention, are useful in that they can inhibit the biological activity of a protein of the present invention.

Usually, the antisense nucleic acids of the present invention include modified oligonucleotides. For example, thioated oligonucleotides can be used to confer nuclease resistance to an oligonucleotide.

Alternatively, the inhibitors of expression of said gene also comprise RNA interferents (interfering RNA or siRNA) compositions (i.e., a composition comprising one or more siRNA oligonucleotides). In the context of the present invention, the siRNA composition reduces the expression of one or more genes selected from the group comprising HOX genes, G13 genes, G18 genes, G25 genes, biologically active fragment thereof and/or combinations thereof.

Herein, the term “RNA interferent” or “siRNA” refers to a double stranded RNA molecule which prevents translation of a target mRNA. In the context of the present invention, the siRNA comprises a sense nucleic acid sequence and an anti-sense nucleic acid sequence against a high expression of one or more genes selected from the group comprising HOX genes, G13 genes, G18 genes, G25 genes, biologically active fragment thereof and/or combinations thereof. The siRNA can be constructed fully synthetically and consisting of two complementary single stranded RNA or biosynthetically The siRNA is constructed such that a single transcript has both the sense and complementary antisense sequences from the target gene, (e.g. a single hairpin RNA or shRNA). Standard techniques for introducing siRNA into the cell can be used, including those in which DNA is a template from which RNA is transcribed.

Usually, an siRNA of one or more genes selected from the group comprising HOX genes, G13 genes, G18 genes, G25 genes, biologically active fragment thereof and/or combinations thereof, hybridizes to target mRNA and thereby decreases or inhibits production of the polypeptides encoded by the genes selected from the group comprising HOX genes, G13 genes, G18 genes, G25 genes, biologically active fragment thereof and/or combinations thereof by associating with the normally single-stranded mRNA transcript, thereby interfering with translation and thus, expression of the protein. Thus, siRNA molecules of the invention can be defined by their ability to hybridize specifically to mRNA or cDNA of one or more genes selected from the group comprising HOX genes, G13 genes, G18 genes, G25 genes, biologically active fragment thereof and/or combinations thereof.

In the context of the present invention, an siRNA is preferably less than 500, preferably less than 200, more preferably less than 100, even more preferably less than 50, or most preferably less than 25 nucleotides in length. More preferably an siRNA is about 19 to about 25 nucleotides in length. In order to enhance the inhibition activity of the siRNA, one or more uridine (“u”) nucleotides can be added to 3′ end of the antisense strand of the target sequence. The number of “u's” to be added is at least 2, generally 2 to 10, preferably 2 to 5. The added “u's” form a single strand at the 3′ end of the antisense strand of the siRNA.

An siRNA of one or more genes selected from the group comprising HOX genes, G13 genes, G18 genes, G25 genes, biologically active fragment thereof and/or combinations thereof, can be directly introduced into the cells in a form that is capable of binding to the mRNA transcripts. In these embodiments, the siRNA molecules of the invention are typically modified as described above for antisense molecules. Other modifications are also possible, for example, cholesterol-conjugated siRNAs have shown improved pharmacological properties. Song, et al, Nature Med. 9:347-51 (2003). Alternatively, a DNA encoding the siRNA can be carried in a vector.

The inhibitors of expression of said one or more genes selected from the group comprising HOX genes, G13 genes, G18 genes, G25 genes, biologically active fragment thereof and/or combinations thereof may also be an antibody, or an immunologically active fragment thereof, that binds to a protein encoded by any one gene selected from the group comprising HOX genes, G13 genes, G18 genes, G25 genes, biologically active fragment thereof and/or combinations thereof.

“Biologically active” means affecting any physical or biochemical properties of a living organism or biological process. Biologically Active Substance refers to any molecule or mixture or complex of molecules that exerts a biological effect in vitro and/or in vivo, including pharmaceuticals, drugs, proteins, peptides, polypeptides, hormones, vitamins, steroids, polyanions, nucleosides, nucleotides, nucleic acids (e.g. DNA or RNA), nucleotides, polynucleotides, etc.

Fragments are sequences sharing at least 40% amino acids in length with the respective sequence of the polypeptide. These sequences can be used as long as they exhibit the same biological properties as the native sequence from which they derive. Preferably these sequences share more than 70%, preferably more than 80%, in particular more than 90% amino acids in length with the respective sequence from which it derives. These fragments can be prepared by a variety of methods and techniques known in the art such as for example chemical synthesis.

A variant is a peptide having an amino acid sequence that differs to some extent from a native sequence peptide, that is an amino acid sequence that vary from the native sequence by conservative amino acid substitutions, whereby one or more amino acids are substituted by another with same characteristics and conformational roles. The amino acid sequence variants possess substitutions, deletions, side-chain modifications and/or insertions at certain positions within the amino acid sequence of the native amino acid sequence. Conservative amino acid substitutions are herein defined as exchanges within one of the following five groups:

I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.

It is to be understood that some non-conventional amino acids may also be suitable replacements for the naturally occurring amino acids. For example Lys residues may be substituted by ornithine, homoarginine, nor-Lys, N-methyl-Lys, N,N-dimethyl-Lys and N,N,N-trimethyl-Lys. Lys residues can also be replaced with synthetic basic amino acids including, but not limited to, N-1-(2-pyrazolinyl)-Arg, 2-(4-piperinyl)-Gly, 2-(4-piperinyl)-Ala, 2-[3-(2S)pyrrolininyl]-Gly and 2-[3-(2S) pyrrolininyl]-Ala. Tyr residues may be substituted with 4-methoxy tyrosine (MeY), meta-Tyr,ortho-Tyr, nor-Tyr,1251-Tyr, mono-halo-Tyr, di-halo-Tyr, O-sulpho-Tyr, O-phospho-Tyr, and nitro-Tyr.

Tyr residues may also be substituted with the 3-hydroxyl or 2-hydroxyl isomers (meta-Tyr or ortho-Tyr, respectively) and corresponding O-sulpho- and O-phospho derivatives. Tyr residues can also be replaced with synthetic hydroxyl containing amino acids including, but not limited to 4-hydroxymethyl-Phe, 4-hydroxyphenyl-Gly, 2,6-dimethyl-Tyr and 5-amino-Tyr.

Aliphatic amino acids may be substituted by synthetic derivatives bearing non-natural aliphatic branched or linear side chains CnH2n+2 where n is a number from 1 up to and including 8. Examples of suitable conservative substitutions by non-conventional amino acids are given in WO02/064740.

Insertions encompass the addition of one or more naturally occurring or non conventional amino acid residues.

Deletion encompasses the deletion of one or more amino acid residues.

Furthermore, since an inherent problem with native peptides (in L-form) is the degradation by natural proteases, the physiological active protein of the invention may be prepared in order to include D-forms and/or “retro-inverso isomers” of the peptide. Preferably, retro-inverso isomers of short parts, variants or combinations of the physiological active protein of the invention are prepared.

Retro-inverso peptides are prepared for peptides of known sequence as described for example in Sela and Zisman, in a review published in FASEB J. 1997 May; 11(6):449-56.

By “retro-inverso isomer” is meant an isomer of a linear peptide in which the direction of the sequence is reversed and the chirality of each amino acid residue is inverted; thus, there can be no end-group complementarity.

The invention also includes analogs in which one or more peptide bonds have been replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic will make the resulting peptide more stable and thus more useful as an active substance. Such mimetics, and methods of incorporating them into peptides, are well known in the art.

The term “inhibitor” or “antagonist” refers to molecules that inhibit the function of the protein or polypeptide by binding thereto.

The term “competitors” refers to “inhibitors” or “antagonists” that directly inhibit the interaction between a protein or polypeptide (i.e. receptor) and its natural ligand resulting in disturbed biochemical or biological function of the receptor. Competitive inhibition is a form of inhibition where binding of the inhibitor prevents binding of the ligand and vice versa. In competitive inhibition, the inhibitor binds to the same active site as the natural ligand, without undergoing a reaction. The ligand molecule cannot enter the active site while the inhibitor is there, and the inhibitor cannot enter the site when the ligand is there.

The “biological activity” of a protein refers to the ability to carry out diverse cellular functions and to bind other molecules specifically and tightly.

The present invention also includes vaccines and vaccination methods. For example, methods of treating or preventing tumor resistance in a subject suffering from a brain tumor can involve administering to the subject a vaccine composition comprising one or more polypeptides encoded by one or more nucleic acids of one or more genes selected from the group comprising HOX genes, G13 genes, G18 genes, G25 genes, biologically active fragment thereof and/or combinations thereof or immunologically active fragments of such polypeptides.

In the context of the present invention, an immunologically active fragment is a polypeptide that is shorter in length than the full-length naturally-occurring protein yet which induces an immune response analogous to that induced by the full-length protein. For example, an immunologically active fragment should be at least 8 residues in length and capable of stimulating an immune cell, for example, a T cell or a B cell Immune cell stimulation can be measured by detecting cell proliferation, elaboration of cytokines (e.g., IL-2), or production of an antibody. See, for example, Harlow and Lane, Using Antibodies: A Laboratory Manual, 1998, Cold Spring Harbor Laboratory Press; and Coligan, et al., Current Protocols in Immunology, 1991-2006, John Wiley & Sons.

Usually the inhibitor of the biological activity of said protein is an antibody or an immunologically fragment thereof that binds to a protein encoded by one or more genes selected from the group comprising HOX genes, G13 genes, G18 genes, G25 genes, biologically active fragment thereof and/or combinations thereof.

Alternatively, function of one or more gene products of the genes over-expressed in cancer can be inhibited by administering a compound that binds to or otherwise inhibits the function of the gene products. For example, the compound is an antibody which binds to the over-expressed gene product or gene products.

As used herein, the term “antibody” refers to an immunoglobulin molecule having a specific structure, that interacts (i.e., binds) only with the antigen that was used for synthesizing the antibody (i.e., the gene product of an up-regulated marker) or with an antigen closely related thereto. Furthermore, an antibody can be a fragment of an antibody or a modified antibody, so long as it binds to one or more of the proteins encoded by the marker genes. For instance, the antibody fragment can be Fab, F(ab′)₂, Fv, or single chain Fv (scFv), in which Fv fragments from H and L chains are ligated by an appropriate linker (Huston J. S. et al., (1988) Proc. Natl. Acad. Sci. U.S.A. 85:5879-83.). More specifically, an antibody fragment can be generated by treating an antibody with an enzyme, including papain or pepsin. Alternatively, a gene encoding the antibody fragment can be constructed, inserted into an expression vector, and expressed in an appropriate host cell (see, for example, Co M. S. et al., (1994) J. Immunol. 152:2968-76; Better M. and Horwitz A. H. (1989) Methods Enzymol. 178:476-96.; Pluckthun A. and Skerra A. (1989) Methods Enzymol. 178:497-515.; Lamoyi E. (1986) Methods Enzymol. 121:652-63.; Rousseaux J. et al, (1986) Methods Enzymol. 121:663-9.; Bird R. E. and Walker B. W. (1991) Trends Biotechnol. 9:132-7.).

An antibody can be modified by conjugation with a variety of molecules, for example, polyethylene glycol (PEG). The present invention provides such modified antibodies. The modified antibody can be obtained by chemically modifying an antibody. Such modification methods are conventional in the field.

Alternatively, an antibody can comprise a chimeric antibody having a variable region from a nonhuman antibody and a constant region from a human antibody, or a humanized antibody, comprising a complementarity determining region (CDR) from a nonhuman antibody, a frame work region (FR) and a constant region from a human antibody. Such antibodies can be prepared by using known technologies. Humanization can be performed by substituting rodent CDRs or CDR sequences for the corresponding sequences of a human antibody {see, e.g., Verhoeyen et ah, (1988) Science 239:1534-6). Accordingly, such humanized antibodies are chimeric antibodies, wherein substantially less than an intact human variable domain has been substituted by the corresponding sequence from a non-human species.

Fully human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom & Winter, (1992) J. MoI. Biol. 227:381-8). Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584; 5,545,807; 5,545,806; 5,569,825; 5,625,126; 5,633,425; 5,661,016. Such antibodies can be prepared by using known technologies.

The present invention also relates to a pharmaceutical composition for the treatment or prevention of a tumor resistance in a subject suffering from a brain tumor, said composition comprising a pharmaceutically effective amount of an antibody or an immunologically fragment thereof that binds to a protein encoded by at least one gene selected from gene clusters comprising HOX genes, G13 genes, G18 genes, G25 genes, biologically active fragment thereof and/or combinations thereof.

The present invention also provides a pharmaceutical composition for the treatment or prevention of a tumor resistance in a subject suffering from a brain tumor, said composition comprising a pharmaceutically effective amount of an RNA antisense comprising a nucleotide sequence complementary to a coding sequence of at least one gene selected from gene clusters comprising HOX genes, G13 genes, G18 genes, G25 genes, biologically active fragment thereof and/or combinations thereof.

The present invention further provides a pharmaceutical composition for the treatment or prevention of a tumor resistance in a subject suffering from a brain tumor, said composition comprising a pharmaceutically effective amount of an RNA interferent.

The present invention further relates to a pharmaceutical composition for the treatment or prevention of a tumor resistance in a subject suffering from the brain tumor, said composition comprising a pharmaceutically effective amount of a compound able to disturb interaction between a protein and a receptor thereof, said protein being encoded by at least one gene selected from gene clusters comprising HOX genes, G13 genes, G18 genes, G25 genes, biologically active fragment thereof and/or combinations thereof.

The present invention also encompasses a pharmaceutical composition for the treatment or prevention of a tumor resistance in a subject suffering from a brain tumor, said composition comprising a pharmaceutically effective amount of a compound obtained with the method of the invention.

“A pharmaceutically effective amount” refers to a chemical material or compound which, when administered to a human or animal organism induces a detectable pharmacologic and/or physiologic effect.

The respective pharmaceutically effect amount can depend on the specific patient to be treated, on the disease to be treated and on the method of administration. Further, the pharmaceutically effective amount depends on the specific protein used, especially if the protein additionally contains a drug as described or not. The treatment usually comprises a multiple administration of the pharmaceutical composition, usually in intervals of several hours, days or weeks. The pharmaceutically effective amount of a dosage unit of the polypeptide usually is in the range of 0.001 ng to 100 mg per kg of body weight of the patient to be treated.

For systemic administration, a therapeutically effective amount or dose can be estimated initially from in vitro assays. For example, a dose can be formulated in animal models to achieve a circulating concentration range that includes the IC50 as determined in cell culture. Such information can be used to more accurately determine useful doses in humans.

Initial doses can also be estimated from in vivo data, e.g. animal models, using techniques that are well known in the art. One ordinarily skill in the art could readily optimise administration to humans based on animal data and will, of course, depend on the subject being treated, on the subject's weight, the severity of the disorder, the manner of administration and the judgement of the prescribing physician.

“Administering”, as it applies in the present invention, refers to contact of the pharmaceutical compositions to the subject, preferably a human.

The pharmaceutical composition may be dissolved or dispersed in a pharmaceutically acceptable carrier well known to those skilled in the art, for parenteral administration by, e.g., intravenous, subcutaneous or intramuscular injection or by intravenous drip infusion.

As to a pharmaceutical composition for parenteral administration, any conventional additives may be used such as excipients, adjuvants, binders, disintegrants, dispersing agents, lubricants, diluents, absorption enhancers, buffering agents, surfactants, solubilizing agents, preservatives, emulsifiers, isotonizers, stabilizers, solubilizers for injection, pH adjusting agents, etc.

Acceptable carriers, diluents and adjuvants which facilitates processing of the active compounds into preparation which can be used pharmaceutically are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the pharmaceutical composition may be systemic or topical. For example, administration of such a pharmaceutical composition may be various parenteral routes such as subcutaneous, intravenous, intradermal, intramuscular, intraperitoneal, intranasal, transdermal, buccal routes or via an implanted device, and may also be delivered by peristaltic means.

The pharmaceutical composition comprising an active ingredient of the present invention may also be incorporated or impregnated into a bioabsorbable matrix, with the matrix being administered in the form of a suspension of matrix, a gel or a solid support. In addition the matrix may be comprised of a biopolymer.

Sustained-release preparations may be prepared. Suitable examples of sustained-release preparations include semi permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished for example by filtration through sterile filtration membranes.

It is understood that the suitable dosage of a peptide of the present invention will be dependent upon the age, sex, health, and weight of the recipient, kind of concurrent treatment, if any and the nature of the effect desired.

The appropriate dosage form will depend on the disease, the protein, and the mode of administration; possibilities include tablets, capsules, lozenges, dental pastes, suppositories, inhalants, solutions, ointments and parenteral depots.

The present invention further relates to a kit for a kit useful for predicting or diagnosing the tumor resistance in a subject treated with concomitant chemo-radiotherapy, said kit comprises a set of primers, probes or antibodies specific for one or more genes selected from gene clusters comprising HOX genes, G13 genes, G18 genes, G25 genes, G07 genes and G14 genes, biologically active fragment thereof and/or combinations thereof.

The cancer-detection reagent of the kit, e.g., a nucleic acid that specifically binds to or identifies one or more nucleic acids associated with tumor resistance to concomitant chemo-radiotherapy, including oligonucleotide sequences which are complementary to a portion of an nucleic acid associated with tumor resistance to concomitant chemo-radiotherapy, or an antibody that binds to one or more proteins encoded by an said nucleic acid. The detection reagents can be packaged together in the form of a kit. For example, the detection reagents can be packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay can also be included in the kit. The assay format of the kit can be a Northern hybridization or a sandwich ELISA, both of which are known in the art. See, for example, Sambrook and Russell, Molecular Cloning: A Laboratory Manual, 3^{rd} Edition, 2001, Cold Spring Harbor Laboratory Press; and Harlow and Lane, Using Antibodies, supra.

Also encompassed in the present invention is a method for screening a candidate compound useful in the treatment or prevention of brain tumor, said method comprising the step of:

a) contacting said candidate compound with a cell expressing one or more genes selected from gene clusters comprising HOX genes, G13 genes, G18 genes, G25 genes, G07 genes and G14 genes, biologically active fragment thereof and/or combinations thereof,
b) selecting the resulting compound that substantially reduces the expression level of one or more genes selected from gene clusters comprising HOX genes, G13 genes, G18 genes, and G25 genes, biologically active fragment thereof and/or combinations thereof, and/or

An agent capable of stimulating the expression of an under-expressed gene or suppressing the expression of an over-expressed gene has clinical benefit. Such agents can be further tested for the ability to prevent cancer in animals or test subjects. As discussed in detail above, by controlling the expression levels of one or more genes of the present invention or the activities of their gene products, one can control the onset and progression of cancer. Thus, candidate agents, which are useful agents in the treatment of cancer, can be identified through screening methods that use such expression levels and activities of as indices of the cancerous or non-cancerous state.

The one or more polypeptides encoded by the genes of the present invention to be used for screening can be a recombinant polypeptide or a protein from the nature or a partial peptide thereof. The polypeptide to be contacted with a test compound can be, for example, a purified polypeptide, a soluble protein, a form bound to a carrier or a fusion protein fused with other polypeptides.

Many methods are known to those skilled in the art can be used for screening for proteins that bind to the one or more cancer polypeptides encoded by the genes of the present invention. Screening can be conducted by, for example, immunoprecipitation methods, specifically, in the following manner. The one or more marker genes are expressed in host (e.g., animal) cells and so on by inserting the gene to an expression vector for foreign genes, for example, pS V2neo, pcDNA I, pcDNA3.1, pCAGGS and pCD8. The promoter to be used for the expression can be any promoter that can be used commonly and include, for example, the SV40 early promoter (Rigby in Williamson (ed.), (1982) Genetic Engineering, vol. 3. Academic Press, London, 83-141.), the EF-[alpha] promoter (Kim et at., (1990) Gene 91: 217-23.), the CAG promoter (Niwa et al, (1991) Gene 108: 193-9.), the RSV LTR promoter (Cullen, (1987) Methods in Enzymology 152: 684-704.) the SRa promoter (Takebe et al., (1988) MoI Cell Biol 8: 466-72.), the CMV immediate early promoter (Seed and Aruffo, (1987) Proc Natl Acad Sci USA 84: 3365-9.), the SV40 late promoter (Gheysen and Fiers, (1982) J MoI Appl Genet. 1: 385-94.), the Adenovirus late promoter (Kaufman et al., (1989) MoI Cell Biol 9: 946-58.), the HSV TK promoter and so on. The introduction of the gene into host cells to express a foreign gene can be performed according to any methods, for example, the electroporation method (Chu et ah, (1987) Nucleic Acids Res 15: 1311-26.), the calcium phosphate method (Chen and Okayama, (1987) MoI Cell Biol 7: 2745-52.), the DEAE dextran method (Lopata et ah, (1984) Nucleic Acids Res 12: 5707-17.; Sussman and Milman, (1984) MoI Cell Biol 4: 1641-3.), the Lipofectin method (Derijard B5 (1994) Cell 76: 1025-37.; Lamb et ah, (1993) Nature Genetics 5: 22-30.: Rabindran et ah, (1993) Science 259: 230-4.) and so on.

The one or more polypeptides encoded by the genes of the present invention can be expressed as a fusion protein comprising a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, [beta]-galactosidase, maltose binding protein, glutathione S-transferase, green florescence protein (GFP) and so on by the use of its multiple cloning sites are commercially available.

A fusion protein prepared by introducing only small epitopes consisting of several to a dozen amino acids so as not to change the property of the polypeptide by the fusion is also reported.

Epitopes, including polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the polypeptide encoded by marker genes (Experimental Medicine 13: 85-90 (1995)).

### EXAMPLES

### Example 1

### Material and Methods

Gene expression profiles of 80 glioblastoma were interrogated for associations with resistance to therapy. Patients were treated within clinical trials testing the addition of concomitant and adjuvant temozolomide to radiotherapy.

### Tumor Samples and Patient Characteristics

Gene expression profiles were established from 80 frozen GBM samples obtained from 76 patients, comprising 70 tumors from initial surgery, and 10 samples resected at recurrence, and from four non-neoplastic brain tissue samples. All patients were treated within a phase II, or a randomized phase III trial {Stupp, 2005 #2126} and provided written informed consent for molecular studies of their tumor. The protocol was approved by the ethics committee at each center. Sixty-eight patients with complete molecular and clinical information were included in survival analysis, with a median age of 51 years (range: 26-70 yrs) at enrollment. Thereof, 42 received TMZ/RT→TMZ, while 26 were randomized to RT only. Second-line therapy frequently involved alkylating agents including TMZ. Patient characteristics are summarized in Table 3. The validation set comprised 76 independent patients of the EORTC/NCIC-study (Stupp et al., 2005), 39 randomized to TMZ/RT→TMZ, and 37 to RT (median age 54 yrs, range 25-69 yrs), whose GBM were available on a tissue microarray. There was no difference in survival as compared to the general trial population, neither in the test population, nor the validation set (P>0.2).

### Gene Expression Profiling

Total RNA was extracted from frozen tumor sections (10-15 mg) (Qiagen, RNeasy-Lipid Tissue Kit). The first section served as reference for diagnosis and tumor content (>70%). Probes were prepared with the ENZO BioArray-HighYield Kit for double amplification and were hybridized to Affymetrix HG-133Plus2.0 GeneChips. The microarray data is deposited in the Gene Expression Omnibus (GEO) database at http://www.ncbi.nml.nih.gov/geo/; (accession-number GSE7696). Quantitative reverse transcription polymerase-chain reaction (qRT-PCR) was performed on the ABI Prism7900 with SYBR Green (Applied Biosystem). Primers are listed in Table 4. Results were normalized to the expression of the EIF2C3, DNAJA4 and B2M genes that exhibit little variation in this dataset.

### Data Analysis and Statistical Methods

Analyses were carried out in R, a free software environment available at http://www.r-project.org/, SAS (V9.1.3), or Coupled Two Way Clustering (CTWC)(Getz et al., 2000) available at: http://ctwc.weizmann.ac.il. The expression intensities for all probe-sets from Affymetrix CEL-files were estimated using robust multi-array average (RMA) with probe-level quantile normalization followed by median polish summarization as implemented in the BioConductor software (http://www.bioconductor.org/).

The expression matrix of 84 samples and 3,860 most variable probe-sets (standard deviation>0.75) was inputed into CTWC using default parameters, and two levels of clustering. CTWC analysis can be viewed at: http://bcf.isb-sib.ch/projects/cancer/glio/. Probe-sets comprised in stable gene clusters emerging from CTWC served as input for supervised analyses.

The Benjamini-Hochberg procedure was applied for multiple testing correction (FDR).

Cox Survival Models Used for Individual Data Sets
Data Sets

| | |
|---|---|
| Hegi42 (TMZ/RT) | y~β₁meanCluster + β₂ MGMT + β₃ |
| | age |
| Hegi42 (TMZ/RT) | y~β₁EGFRvIII + β₂ MGMT + β₃ age + β₄ EGFRwt |
| Hegi42 (TMZ/RT) | y~β₁meanG98 + β₂ EGFR + β₃ MGMT + β₄ age |
| Hegi68 (all, G98) | y~β₁meanG98 + β₂ MGMT + β₃ age + β₄ TMZ + |
| | β₅ meanG98:TMZ + β₆ MGMT:TMZ |
| Hegi68 (all, EGFR) | y~β₁ EGFR + β₂ MGMT + β₃age + β₄ TMZ + |
| | β₅ EGFR:TMZ + β₆ MGMT:TMZ |
| Nelson | y~β₁meanCluster + β₂ grade + β₃ age |
| Aldape | y~β₁meanCluster + β₂ grade + β₃ age |
| combined | y~β₁meanCluster + β₂ grade + β₃ age + |
| | strata (dataSet) |

MGMT, methylation status
MGMT:TMZ, interaction factor with TMZ/RT→TMZ therapy

### Partial Least Square (PLS)

Partial Least Square (PLS) regression is a technique that combines features from principal component analysis and from multiple linear regression ^{s}. It is particularly useful to predict an outcome from a large set of highly correlated predictors. In the present invention, the PLS procedure in SAS was used to define combinations of the genes expressions (i.e. factors) which attempt to explain the genes expressions variability and the survival outcome at the same time. For each patient, the martingale residuals obtained from a Cox regression with a constant as the only predictor were used as outcome variable to be explained ^{s}. This approach was derived from the works of Therneau et al. ^{s} and Leblanc et al. ^{s} with the CART algorithm and applied to PLS regression. This allowed to account for the effect of censoring on survival estimates.

The optimal number of PLS factors is generally obtained by cross validation, but in this small sample (n=42 or 68) this method was not conclusive (i.e. no optimal number of factors could be obtained). Therefore, we empirically chose to fit a PLS model with two factors to minimize overfitting and get interpretable results. The two factors explained 66% of the survival outcome variations. The contribution of each gene was assessed by their coefficients in the linear regression and their X-weights plotted in a factorial plane. Arbitrarily, clusters were given X-weights by averaging the X-weights of their constituent genes.

### Tissue Micro Array (TMA) and Immunohistochemistry

The TMA was constructed with glioblastoma from the EORTC/NCIC-trial (Stupp et al., 2005). HOXA10 (Santa Cruz, sc-17159; dilution 1:200) expression was determined by immunohistochemistry (citrate buffer ph 6.0, 15 min pressure cooker) and scored without knowledge of clinical information on a scale of 0 to 6 (0, no expression; 1, weak nuclear expression in <20% cells; 2, strong in <20% cells; 3, weak in 20 to 50% cells; 4, strong in 20 to 50% cells; 5, weak in >50% cells; 6, strong in >50% cells). Dichotomization for survival analysis was no-low (0,1) versus intermediate-high expression (2-6). Frozen sections of neurospheres were fixed with acetone and stained for HOXA10 and CD133 (Santa Cruz, sc-17159, dilution 1:100; Miltenyi AC133-2, 293C3, dilution 1:50).

### Glioblastoma Derived Neurospheres

Fresh glioblastoma tissue was dissociated in presence of papain and DNase I basically as described (Clement et al., 2007). Cells were cultured under stem cell conditions to form spheres using Dulbecco's modified Eagle medium/F12 medium containing B27 supplement and 20 ng/ml of both EGF and FGF2. Neurospheres from 6 glioblastoma propagated between 4 weeks and 14 months were used for immunostaining.

### Description of External Data Sets

Two external gene expression data sets (GeneChip U133A & B, Affymetrix) of malignant glioma were used for validation of survival factors, referred to as “Nelson-” ^{s}(Freije et al., 2004) and “Aldape-” data sets ^{s}(Phillips et al., 2006), respectively. Only cases with diagnosis of glioblastoma, astrocytoma grade III, and mixed-oligoastrocytoma grade III were included. The Nelson data set utilized here comprised 17 grade III and 54 grade IV glioma. The median age was 43 years at diagnosis for all 71 patients, or 51 years for GBM patients, respectively. The Aldape data set consisted of 75 patients, 27 grade III, 48 grade IV with a median age at diagnosis of 47 years for all cases, or 49 years for GBM patients, respectively. This data set was enriched for long term survivors according to the authors. Adjustments in the Cox models for these two external glioma validation sets comprised age as a dichotomous variable (>50 years) and tumor grade.

In addition, we used a mouse expression data set published by Krivtsov et al. ^{s}(Krivtsov et al., 2006) and a human leukemia data set by Ross et al. ^{s}(Ross et al., 2004) Table 9. For both human malignant glioma validation sets, only the chips that passed quality criteria based on the BioConductor library affyPLM ^{s} were included. The external data sets were each separately normalized using RMA, when CEL-files were available (Aldape, Nelson and Ross). For the Krivtsov mouse expression data set ^{s}(Krivtsov et al., 2006), the Affymetrix Microarray Suite version 5.0 processed data, as submitted in the Gene Expression Omnibus (GEO) database, were used. For the human-murine comparison, we mapped the human gene sets to homolog murine gene sets using the annotation provided by Affymetrix on their webpage http//www.affymetrix.com (version of Nov. 14, 2006). For GSEA analysis the data sets were filtered to retain only the most variant probe sets (standard deviation greater than 0.5).

### Supervised Analysis

For each covariate, the hazards ratio, its 95% confidence interval and the associated Wald p-value were examined The Benjamini Hochberg procedure ^{s} was applied as multiple testing correction method to convert p-values into estimated false discovery rates (FDR). The hazards ratio for a gene (or the mean of a cluster) were standardized by referring them to a variation corresponding to an interquartile range, in order to obtain an interpretable value, for microarray as well as for real-time PCR data. Therefore, the interpretation of an estimated hazard ratio of 1.5 is that the hazard rate increases by 50 percent for an increase of one interquartile range of the log-scale gene expression, independently of the expression level at which it is calculated.

### Example 2

### Results

An expression signature dominated by HOX-genes which comprises Prominin-1 (CD133), emerged as a predictor for poor survival in patients treated with concomitant chemo-radiotherapy (n=42; hazard ratio 2.69, 95% CI 1.38-5.26, P=0.004). This association could be validated in an independent data-set. Provocatively, the HOX-gene cluster was reminiscent of a “self-renewal” signature (P=0.008; Gene Set Enrichment Analysis). The HOX-gene signature and EGFR-gene expression were independent prognostic factors in multivariate analysis, adjusted for the MGMT-methylation status, a known predictive factor for benefit from temozolomide, and age. Better outcome was associated with gene clusters characterizing features of tumor host interaction including tumor vascularization and cell adhesion, and innate immune response.

Gene Expression Signatures Associated with Tumor Resistance There was no obvious association of patient characteristics or survival with genetic subtypes evident from the sample dendrogram S1(G1), clustering all samples (S1) and all genes (G1) passing a variation filter (FIG. 1A). The methylation status of the MGMT gene promoter appears to be randomly distributed. All stable gene clusters emerging from this analysis are listed in Table 1, named by the predominant biological function suggested by the genes they comprise, while their inter-relationship is visualized in FIG. 1B.

Similar gene clusters are obtained when using only the subset of glioblastoma clustered in S4 [G1(54)] (Table 5).

Cluster S4 comprises most glioblastoma (69/80), but not the non-tumoral tissues that form their own stable cluster S3 (FIG. 1A). Similar gene clusters are present in other glioblastoma data-sets as visualized in FIG. 1C for data-sets published by the group of Nelson and Aldape, respectively (Freije et al., 2004; Phillips et al., 2006). All eighteen non-overlapping, stable gene clusters [G1(S1)] were interrogated for association with survival using Cox proportional hazards, adjusted for age (>50 years) and MGMT methylation status (Hegi et al., 2005). Seven gene clusters were most influential for explaining survival in patients randomized to TMZ/RT→TMZ (Table 1; gene lists, Tables A8-A13). A respective PLS-model yielded comparable results (FIG. 5). However, the MGMT methylation status was yet the most influential predictor of survival (Table 2, FIG. 5). In this invention, the Applicants identified and focused on the two most significant clusters characterized by HOX (G28/G98 clusters) and EGFR gene expression (G25 clusters) and other relevant gene clusters presented in the present invention.

“Self-Renewal Signature” Associated with Resistance to Chemo-Radiotherapy in Glioblastoma

Increased expression of cluster G28, dominated by HOX-genes and comprising the cell-cycle checkpoint gene GADD45G (Vairapandi et al., 2002), was found to be associated with worse outcome (P=0.004; HR 2.69, 95% CI 1.38-5.26; Table 1). The interaction term between this HOX-gene cluster and chemo-radiotherapy was significant (P=0.001) in the Cox model, when evaluating all 68 patients from the two treatment arms, implying that high expression of HOX-genes may be predictive for resistance to TMZ/RT→TMZ therapy.

The HOX gene clusters G28 and G98 emerging from clustering all genes (G1) either with all samples (S1) or only with glioblastoma clustered in S4, are almost identical (Pearson correlation0.98; 19/21 probe-sets, Table 7).

Intriguingly, G98 in addition comprises PROM1 (prominin 1) encoding the putative glioma stem cell marker CD133 (FIG. 2A), suggesting that in a subpopulation of glioblastoma concerted upregulation of HOX-genes might be associated with a tumor stem-like cell phenotype. In accordance, we find high HOXA10 protein expression in glioblastoma derived neurospheres, cultured under stem cell conditions, as displayed in FIG. 2B together with CD133 expression. To include information on PROM1 the Applicant's show results on G98 for all following analyses. FIG. 2C visualizes the association of short survival with enhanced expression of G98.

### Validation in Independent Datasets

The association of the HOX-signature with resistance to treatment was subsequently validated in a sample set of the trial not available for initial discovery, arrayed on a TMA. HOXA10 was evaluated by immunohistochemistry (FIG. 3A) as a representative of the correlated set of HOX-genes. Strong nuclear expression was often observed in patches of tumor cells, situated in the vicinity of blood vessels. High HOXA10 expression was associated with worse outcome in patients randomized to TMZ/RT→TMZ therapy (n=39, HR 2.57, 95% CI 1.21-5.47, p=0.014) (FIG. 3B). The patients randomized to RT only did not show such a relationship, suggesting that high HOXA10 expression may be predictive for resistance to a synergistic effect of concomitant chemo-radiotherapy, in concordance with the significant interaction term between treatment and expression of G28 or G98 (Table 6).

Similar HOX-gene clusters can be identified in the Nelson and Aldape glioblastoma datasets (Freije et al., 2004; Phillips et al., 2006). Correlating G98 gene expression with outcome in these datasets totaling 102 glioblastoma revealed a trend for worse outcome (P=0.09, HR 1.29, 95% CI 0.97-1.72) (FIG. 6). Of note, in contrast to our data, these patients were treated before the TMZ/RT→TMZ regimen was established. In accordance with better survival, anaplastic glioma (WHO grade III) profiled in these publications revealed significantly lower expression of G98 genes as compared to glioblastoma (WHO grade IV) (P<0.001 Aldape data-set (Freije et al., 2004); P=0.002, Nelson data-set (Phillips et al., 2006), Wilcoxon rank sum test with continuity correction, FIG. 7). However, within grade III glioma of the two data-sets increased expression of G98 genes was associated with worse outcome (n=44, p=0.007, HR 3.35, 95% CI 1.39-8.08, FIG. 6).

### HOX-Signature Reminiscent of “Self-Renewal”

The Applicant's G98-derived signature was significantly enriched in genes discriminating “self-renewal” versus “non-self-renewal” in this expression dataset according to Gene Set Enrichment Analysis (GSEA) (Subramanian et al., 2005) (P=0.008) (FIG. 8) and the Wilcoxon two-sample test (G98, P<0.001). The relevance of our signature was further demonstrated in a human dataset (Ross et al., 2004) in which G98, similar to the original murine “self-renewal signature” (Krivtsov et al., 2006), was able to significantly differentiate MLL rearranged AML from AML (GSEA, P<0.001; Wilcoxon two-sample test, P=0.01) Table 9.

Interestingly, a significant, though low correlation between the mean DNA copy number of the two BAC (bacterial artificial chromosome) clones (GS1-213H12 and CTB-23D20) bordering the HOXA gene locus on chromosome 7 and the mean expression of the HOXA genes was observed in a set of 60 glioblastoma (Pearson correlation coefficient r=0.27, P=0.03) (manuscript in preparation). These flanking BACs were more amplified than their neighbors (FIG. 9). Hence, the here HOX-dominated “self-renewal signature” for glioblastoma may in part be acquired by increased gene dosage.

High EGFR Expression is Associated with Tumor Resistance.

Two gene clusters representing amplification-mediated overexpression of proto-oncogenes were associated with tumor resistance: G13 (P=0.02, HR 1.1, 95% CI 1.0-1.2) characterized by coordinated upregulation of contiguous genes on chromosome 12q13-15, comprising the proto-oncogenes CDK4 and MDM2, and G25, dominated by EGFR probesets (P=0.002, HR, 2.8, 95% CI 1.4-5.4) (Table 1, FIG. 4). The array-derived EGFR-measurement (probeset 201983_s_at) was confirmed by qRT-PCR (Pearson correlation 0.89). Expression of the constitutively activated EGFRvIII mutant (18/70; 26%), as measured by qRT-PCR, did not further influence outcome prediction (P=0.94).

Cluster G18, associated with tumor resistance (P=0.03; HR 1.94, 95% CI 1.07-3.51), displayed some correlation with EGFR expression (r=0.57) (FIG. 1B) in particular with Aquaporin 4 (AQP4). AQP4 has been associated with brain tumor related edema (Manley et al., 2000). Aquaporins require activation of mitogen-activated protein kinase (MAPK) signalling that may be mediated by EGFR activation (Herrlich et al., 2004). Another family member, AQP1, has been linked with tumor angiogenesis and cell migration (Saadoun et al., 2005) and was associated with worse outcome in the present invention (P=0.003; HR: 2.44, 95% CI: 1.36-4.04) and in the two external datasets (combined P=0.009; HR=1.51; 95% CI 1.11-2.06).

Blood Vessels Markers Associated with Better Outcome.

G7 is characterized by genes associated with endothelial cells, basement membranes, signaling pathways of vascular development and angiogenesis, and tumor-derived endothelial markers (Madden et al., 2004; St Croix et al., 2000) (Table 10).

Hence, G7 may differentiate tumors according to their angiogenic pattern that may be indicative for drug perfusion and therefore show association with benefit from treatment (Table 1). G7 is in the center of a “super cluster” (FIG. 1B) constituted of several stable gene clusters with biologically related features, such as hypoxia regulated genes G9, and the myeloid progenitor/adhesion cluster G14, which is also correlated with better outcome (Table 1). Beside genes related to cell adhesion, mesenchymal stem cells (PRR16), and homeobox genes (MEOX1, MKX), G14 includes aldehyde dehydrogenase (Chute et al., 2006) and bone morphogenetic protein 5 (BMP5) (Piccirillo et al., 2006) both associated with differentiation of stem-cells. The cluster comprises positive (MEOX1) and negative regulators (SOSTDC1) of BMPs, which recently have been shown to inhibit tumorigenic potential of human brain tumor stem cells by promoting their differentiation (Piccirillo et al., 2006). This cluster may reflect the perivascular microenvironment proposed recently to serve as niche for brain tumor stem cells (Calabrese et al., 2007).

Innate Immune Response Associated with Better Survival.

Alongside markers of innate immunity and macrophages (CD11b), G24 comprises numerous cell surface receptors known as markers for M2 polarized macrophages (Mantovani et al., 2002), such as CD163 (Table 14). M2-polarization mediates tolerance and down-regulates inflammation, alleviating immune surveillance (Mantovani et al., 2002). A wide range of CD163 positive cells can be observed in glioblastoma (FIG. 10). The cluster also contains probes encoding MHC class II surface molecules, but lacks expression of co-stimulatory molecules critical for T-cell activation. This immune signature may be relevant for strategies of tumor vaccination.

### HOX-Signature and EGFR Expression are Independent Prognostic Factors.

Multivariate analysis suggests that the HOX-signature and EGFR expression, respectively, are independent prognostic factors for poor outcome in TMZ/RT→TMZ treated glioblastoma patients, explaining 67% of the survival outcome variations together with the MGMT-status and age (Table 2).

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

TABLE 1
Cox Analysis Using Stable Gene Clusters Identified in G1(S1) in the Cohort of
Treated Patients (n = 42)

| | | | # | | | | | |
|---|---|---|---|---|---|---|---|---|
| | | | Probes | | | | | |
| From G1 (S1) | | | in | ^{a}P- | | | | |
| Main Clusters | Subclusters | Description | Cluster | Value | ^{b}FDR | ^{c}HR | 95% CI | |
| G2 | | Migration-related | 19 | 0.21 | 0.32 | 0.73 | 0.45 | 1.19 |
| G7 | | Tumor Blood Vessel Markers | 77 | 0.04 | 0.11 | 0.73 | 0.54 | 0.99 |
| G9 | | Hypoxia-induced | 32 | 0.70 | 0.78 | 0.89 | 0.49 | 1.61 |
| G11 | | SOX Genes | 15 | 0.25 | 0.34 | 1.46 | 0.77 | 2.77 |
| G12 | | Interferon-induced Genes | 80 | 0.78 | 0.83 | 1.06 | 0.72 | 1.55 |
| G13 | | Chromosome 12 | 39 | 0.02 | 0.09 | 1.13 | 1.02 | 1.24 |
| G14 | | Myeloid Lineage/Adhesion | 26 | 0.03 | 0.09 | 0.61 | 0.39 | 0.95 |
| G16 | | Glial Lineage | 49 | 0.07 | 0.16 | 1.64 | 0.96 | 2.81 |
| G18 | | Brain Physiology | 25 | 0.03 | 0.09 | 1.94 | 1.07 | 3.51 |
| G20 | | ?? | 24 | 0.20 | 0.32 | 1.36 | 0.85 | 2.19 |
| G21 | >G17 > G10 > G3 | Neural Genes | 213 | 0.99 | 0.99 | 1.00 | 0.69 | 1.46 |
| G22 | | Imprinted Genes | 34 | 0.20 | 0.32 | 0.65 | 0.34 | 1.25 |
| G23 | >G19 > G4 | Oligodendrocyte Markers | 110 | 0.66 | 0.78 | 1.07 | 0.78 | 1.47 |
| G24 | >G15 > G8 | Innate Immune Response | 134 | 0.03 | 0.09 | 0.65 | 0.44 | 0.96 |
| G25 | | EGFR | 18 | 0.002 | 0.03 | 2.78 | 1.44 | 5.36 |
| G27 | >G5 | Spermatogenesis | 79 | 0.10 | 0.20 | 1.38 | 0.94 | 2.03 |
| G28 | | HOX Genes | 20 | 0.004 | 0.03 | 2.69 | 1.38 | 5.26 |
| G29 | >G26 > G6 | Proliferation | 101 | 0.43 | 0.55 | 1.27 | 0.70 | 2.32 |

TABLE 2
Overall Cox Proportional-Hazards Models for the Cohort of 42 Patients
Treated with TMZ/RT→TMZ

| | Univariate Model | | | Multivariate Model | | |
|---|---|---|---|---|---|---|
| | Hazard Ratio | | | Hazard Ratio | | |
| Variables | (95% CI) | P-Value | R² | (95% CI) | P-Value | R² |
| HOX cluster (G98), mean | 1.89 (1.03-3.46) | 0.04 | 0.10 | 3.32 (1.61-6.82) | 0.001 | ^{a}0.67 |
| expression | | | | | | |
| EGFR expression | 1.87 (1.09-3.22) | 0.02 | 0.12 | 3.13 (1.62-6.04) | <0.001 | |
| MGMT-methylation | 0.15 (0.06-0.37) | <0.001 | 0.38 | 0.06 (0.0-0.20) | <0.001 | |
| Age >50 years | 1.89 (0.93-3.83) | 0.08 | 0.07 | 2.61 (1.22-5.57) | 0.01 | |

HR, Hazard Ratio;
CI, confidence interval
^{a}R² for the overall multivariate Cox Proportional-Hazards Model.

TABLE 3
Summary of patient information

| | Patient | | | MGMT | OS | | | |
|---|---|---|---|---|---|---|---|---|
| Tissue^{a} | Id | Rec Id | Gender | status | months | Age | Treatment^{b} | Censor |
| GBM | 12 | | M | M | 17 | 38 | TMZ/RT→TMZ | uncensored |
| GBM | 15 | | F | U | 6 | 45 | RT | uncensored |
| GBM | 30 | | M | M | 7 | 61 | TMZ/RT→TMZ | uncensored |
| GBM | 55 | | M | M | 10 | 50 | RT | uncensored |
| GBM | 57 | 57R | M | M | 36 | 53 | TMZ/RT→TMZ | censored |
| GBM | 63 | | M | M | 16 | 49 | RT | uncensored |
| RecGBM | | 65R | F | M | 13 | 46 | TMZ/RT→TMZ | uncensored |
| GBM | 70 | | M | ? | 34 | 53 | RT | censored |
| GBM | 80 | | F | M | 30 | 47 | TMZ/RT→TMZ | uncensored |
| GBM | 83 | | M | U | 16 | 49 | RT | uncensored |
| GBM | 84 | | M | M | 1 | 54 | RT | uncensored |
| GBM | 90 | | F | M | 30 | 61 | TMZ/RT→TMZ | censored |
| GBM | 93 | | M | U | 10 | 70 | TMZ/RT→TMZ | uncensored |
| GBM | 94 | | M | U | 9 | 59 | RT | uncensored |
| GBM | 97 | | F | U | 4 | 62 | RT | uncensored |
| RecGBM | | 100R | M | U | 24 | 64 | TMZ/RT→TMZ | uncensored |
| GBM | 104 | | M | M | 20 | 56 | TMZ/RT→TMZ | uncensored |
| GBM | 106 | | M | U | 10 | 27 | TMZ/RT→TMZ | uncensored |
| GBM | 129 | | M | U | 4 | 49 | TMZ/RT→TMZ | uncensored |
| GBM | 147 | | M | M | 15 | 49 | RT | uncensored |
| norm. brain | 148 | | | | | | | NA |
| RecGBM | | 155R | M | M | 21 | 52 | TMZ/RT→TMZ | uncensored |
| GBM | 157 | | M | U | 8 | 49 | TMZ/RT→TMZ | uncensored |
| GBM | 159 | | F | U | 14 | 61 | RT | uncensored |
| GBM | 161 | | M | U | 12 | 53 | TMZ/RT→TMZ | uncensored |
| GBM | 190 | | M | M | 17 | 61 | TMZ/RT→TMZ | uncensored |
| GBM | 211 | | M | M | 9 | 44 | RT | uncensored |
| RecGBM | | 212R | F | U | 16 | 59 | RT | uncensored |
| GBM | 218 | | F | M | 16 | 48 | RT | uncensored |
| GBM | 222 | | F | U | 12 | 62 | TMZ/RT→TMZ | uncensored |
| GBM | 236 | | M | U | 11 | 57 | TMZ/RT→TMZ | uncensored |
| GBM | 242 | | M | U | 14 | 54 | RT | uncensored |
| GBM | 246 | | M | M | 23 | 54 | RT | uncensored |
| GBM | 254 | | M | U | 7 | 64 | RT | uncensored |
| GBM | 269 | | M | U | 7 | 61 | TMZ/RT→TMZ | uncensored |
| GBM | 274 | | F | U | 9 | 49 | TMZ/RT→TMZ | uncensored |
| GBM | 283 | | M | M | 7 | 57 | RT | uncensored |
| GBM | 284 | | M | M | 27 | 52 | RT | uncensored |
| GBM | 288 | | M | M | 27 | 45 | TMZ/RT→TMZ | censored |
| GBM | 300 | | M | ? | 9 | 47 | TMZ/RT→TMZ | uncensored |
| GBM | 323 | | M | M | 16 | 53 | RT | uncensored |
| GBM | 328 | | M | U | 11 | 63 | RT | uncensored |
| GBM | 344 | | M | U | 16 | 57 | TMZ/RT→TMZ | uncensored |
| GBM | 345 | | M | M | 6 | 59 | RT | uncensored |
| GBM | 349 | | F | M | 14 | 52 | RT | uncensored |
| GBM | 356 | | M | U | 7 | 32 | TMZ/RT→TMZ | uncensored |
| GBM | 360 | | F | M | 23 | 56 | TMZ/RT→TMZ | censored |
| GBM | 390 | | M | U | 16 | 42 | RT | uncensored |
| GBM | 396 | | M | M | 16 | 50 | TMZ/RT→TMZ | censored |
| GBM | 428 | | M | M | 9 | 57 | RT | uncensored |
| GBM | 444 | | M | M | 6 | 63 | TMZ/RT→TMZ | uncensored |
| GBM | 458 | | F | U | 14 | 50 | TMZ/RT→TMZ | uncensored |
| GBM | 461 | | M | U | 15 | 64 | TMZ/RT→TMZ | uncensored |
| GBM | 506 | | M | U | 6 | 57 | TMZ/RT→TMZ | uncensored |
| GBM | 527 | | F | M | 24 | 53 | RT | censored |
| GBM | 528 | | M | U | 24 | 33 | TMZ/RT→TMZ | censored |
| GBM | 534 | | F | M | 23 | 42 | RT | censored |
| GBM | 547 | | M | U | 17 | 43 | TMZ/RT→TMZ | uncensored |
| GBM | 558 | | F | U | 6 | 56 | TMZ/RT→TMZ | uncensored |
| GBM | 563 | | M | U | 9 | 53 | TMZ/RT→TMZ | uncensored |
| GBM | 566 | | M | U | 16 | 44 | RT | uncensored |
| GBM | 574 | | F | U | 5 | 44 | RT | uncensored |
| norm. brain | 1076 | | | | | | | NA |
| GBM | 1284 | | F | M | 47 | 48 | TMZ/RT→TMZ | uncensored |
| GBM | 1297 | | M | M | 60 | 36 | TMZ/RT→TMZ | uncensored |
| GBM | 1308 | | F | M | 17 | 45 | TMZ/RT→TMZ | uncensored |
| GBM | 1317 | | M | U | 17 | 26 | TMZ/RT→TMZ | uncensored |
| GBM | 1357 | | M | M | 28 | 36 | TMZ/RT→TMZ | uncensored |
| GBM | 1360 | | M | M | 9 | 65 | TMZ/RT→TMZ | uncensored |
| GBM | 1399 | | M | M | 79 | 39 | TMZ/RT→TMZ | censored |
| GBM | 1430 | 1497R | M | M | 15 | 37 | TMZ/RT→TMZ | uncensored |
| GBM | 1437 | | M | U | 16 | 48 | TMZ/RT→TMZ | uncensored |
| GBM | 1453 | | F | U | 9 | 53 | TMZ/RT→TMZ | uncensored |
| norm. brain | 1553 | | | | | | | NA |
| GBM | 1621 | 1622R | M | M | 25 | 62 | TMZ/RT→TMZ | uncensored |
| norm. brain | 1780 | | | | | | | NA |
| RecGBM | 1795R | 1869RR | M | M | 72 | 43 | TMZ/RT→TMZ | censored |
| RecGBM | | 1854R | M | M | 63 | 52 | TMZ/RT→TMZ | censored |
| GBM | ge197 | | M | M | 53 | 46 | TMZ/RT→TMZ | censored |
| GBM | ge205 | | F | M | 23 | 56 | TMZ/RT→TMZ | uncensored |

TABLE 4
Primer Sequences Used in qRT-PCR

| | | | SEQ |
|---|---|---|---|
| | | | ID |
| Target | | Primer Sequences | N^{o} |
| EIF2C3 | Forward | 5′-CTACATTGTAGTTCAGAAGAGACATC | 1 |
| | primer | ACA-3′ | |
| | Reverse | 5′-TGCCACTTCTTCCAACCCTTT-3′ | 2 |
| | primer | | |
| DNAJA4 | Forward | 5′-GGATGAGGGCGAGAAGTTTAAA-3′ | 3 |
| | primer | | |
| | Reverse | 5′-GGTCATAAACATCCCTTTTCTTTGG-3′ | 4 |
| | primer | | |
| B2M | Forward | 5′-TGCTCGCGCTACTCTCTCTTT-3′ | 5 |
| | primer | | |
| | Reverse | 5′-TCTGCTGGATGACGTGAGTAAAC-3′ | 6 |
| | primer | | |
| EGFRwt | Forward | 5′-GTGGTCCTTGGGAATTTGGA-3′ | 7 |
| | primer | | |
| | Reverse | 5′-CACCTCCTGGATGGTCTTTAAGA-3′ | 8 |
| | primer | | |
| EGFRvIII | Forward | 5′-CGCTGCTGGCTGCGCTCTG-3′ | 9 |
| | primer | | |
| | Reverse | 5′-AGATGGAGGAAGACGGCGT-3′ | 10 |
| | primer | | |

TABLE 5
Cox analysis using G1 (S4) clusters in the TMZ/RT treated patient cohort (n = 42)

| | Stable Clusters in G1 (S4) | | | | | | | | |
|---|---|---|---|---|---|---|---|---|---|
| ^{a}S1vs S4 | Main | | | | | | | | |
| clusters | Clusters | Subclusters | Description | # probes | ^{b}p-value | ^{c}FDR | ^{d}HR | 95% CI | |
| pG02 | G71 | | Astrocyte Markers (?) | 17 | 0.03 | 0.11 | 0.58 | 0.36 | 0.96 |
| pG02/pG07 | G72 | | Migration-related | 17 | 0.29 | 0.49 | 0.76 | 0.46 | 1.26 |
| G23 | G74 | | Oligodendrocyte Markers | 79 | 0.67 | 0.87 | 1.07 | 0.79 | 1.44 |
| G24/pG07 | G80 | | Innate Immune Response | 152 | 0.03 | 0.11 | 0.62 | 0.41 | 0.94 |
| G29 | G82 | >G77 | Proliferation | 98 | 0.47 | 0.72 | 1.26 | 0.67 | 2.36 |
| G13 | G83 | | Chromosome 12 | 37 | 0.02 | 0.11 | 1.08 | 1.01 | 1.15 |
| G09 | G84 | | Hypoxia-induced Genes | 54 | 0.51 | 0.72 | 0.83 | 0.47 | 1.45 |
| | G88 | | ? TGF-beta, myc, SOD2 | 20 | 0.08 | 0.22 | 0.68 | 0.44 | 1.05 |
| G07 pG14 | G90 | >G79 > G73; G85 | Tumour Blood Vessel Markers/Adhesion | 131 | 0.01 | 0.10 | 0.60 | 0.40 | 0.89 |
| pG16/pG29 | G91 | >G86 | Membrane & Cytoskeleton Proteins | 89 | 0.91 | 0.94 | 0.96 | 0.52 | 1.79 |
| pG16 | G92 | | Protein binding, glut metab., OLIG (?) | 93 | 0.14 | 0.29 | 1.49 | 0.88 | 2.54 |
| | G93 | | Regulation of Transcription, EGRs | 20 | 0.75 | 0.91 | 0.93 | 0.59 | 1.45 |
| G21 | G94 | >G89 > G78 | Neural Genes | 168 | 0.94 | 0.94 | 0.98 | 0.59 | 1.63 |
| G20 | G95 | | ?? = G20 | 19 | 0.25 | 0.48 | 1.39 | 0.79 | 2.44 |
| G12 | G96 | >G87 > G81 > G75 | Interferon-induced Genes | 75 | 0.82 | 0.93 | 1.04 | 0.73 | 1.48 |
| G27 | G97 | >G76 | Spermatogenesis | 78 | 0.09 | 0.22 | 1.38 | 0.95 | 2.00 |
| G28 | G98 | | HOX Genes | 21 | 0.004 | 0.07 | 2.56 | 1.34 | 4.88 |

HR, hazard ratio;
CI, confidence interval.
^{a} corresponding clusters in G1 (S1); p, partial G1 (S1) cluster
^{b}p-value for the coefficient relative to the mean of the cluster in a multivariate Cox proportional hazards model adjusted for MGMT methylation status and age as additive independent risk factors (y~ β₁meanCluster + β₂ MGMT + β₃ age)
^{c}FDR, False Discovery Rate
^{d}each continuous variable was scaled to have the interquartile range equal to 1 and median equal to 0.

TABLE 6
Estimated Hazard Ratios (HR) for Treatment Effect in
MGMT-methylated Patients at Different Expression Levels
of G98 Multivariate Proportional Hazards Cox Model with
N = 68.

| | | | P-value |
|---|---|---|---|
| | | | Treatment |
| Expression Levels | HR | 95% CI | Interaction Term |
| G98 | | | 0.002 |
| ^{1st} quartile | 0.13 | 0.05-0.37 | |
| median | 0.25 | 0.11-0.60 | |
| 3^{rd} quartile | 0.47 | 0.20-1.13 | |

The log-hazard function was defined as:

g(t,X,TMZ,MGMT,age,β)=ln [h0(t)]+β₁X+β₂TMZ+β₃MGMT+β₄age+β₅(X*TMZ)+β₆(TMZ*MGMT)

where X represents the expression level of G98 (summarized as the mean of the cluster), tmz is the dichotomous variable representing the regimen chemoradiotherapy versus radiotherapy alone, MGMT is a dichotomous variable representing MGMT promoter methylation status, age is a dichotomous variable (>50 years at the time of randomization).

TABLE 7
G28 versus G98 probesets

| G1 (S1) | G1 (S4) | Probeset | RefSeq.Transcript ID | |
|---|---|---|---|---|
| G28 | — | 204121_at | NM_006705 | GADD45G Chr: 9q22.1-q22.2 |
| G28 | G98 | 1557051_s_at | — | — |
| G28 | G98 | 204362_at | NM_003930 | SCAP2 Chr: 7p21-p15 |
| G28 | G98 | 205522_at | NM_014621 | HOXD4 Chr: 2q31.1 |
| | | | NM_004503 /// | |
| | | | NM_014620 /// | |
| | | | NM_153633 /// | |
| G28 | G98 | 206858_s_at | NM_153693 | HOXC6 Chr: 12q13.3 |
| G28 | G98 | 209905_at | NM_152739 | HOXA9 Chr: 7p15-p14 |
| | | | NM_018951 /// | |
| G28 | G98 | 213150_at | NM_153715 | HOXA10 Chr: 7p15-p14 |
| G28 | G98 | 213844_at | NM_019102 | HOXA5 Chr: 7p15-p14 |
| G28 | G98 | 214651_s_at | NM_152739 | HOXA9 Chr: 7p15-p14 |
| G28 | G98 | 225639_at | NM_003930 | SCAP2 Chr: 7p21-p15 |
| G28 | G98 | 226582_at | — | LOC400043 chr12q13.13 |
| | | | XM_001716150 /// | |
| | | | XM_374020 /// | |
| G28 | G98 | 228564_at | XM_944366 | LOC375295 Chr: 2q31.2 |
| G28 | G98 | 228642_at | — | — |
| G28 | G98 | 229400_at | NM_002148 | HOXD10 Chr: 2q31.1 |
| G28 | G98 | 231906_at | NM_019558 | HOXD8 Chr: 2q31.1 |
| | | | NM_030661 /// | |
| | | | NM_153631 /// | |
| G28 | G98 | 235521_at | NM_153632 | HOXA3 Chr: 7p15-p14 |
| G28 | G98 | 235753_at | NM_006896 | HOXA7 chr7p15-p14 |
| G28 | G98 | 238847_at | — | HOXD10 chr2q31.1 |
| G28 | G98 | 239153_at | — | FLJ41747 chr12q13.13 |
| G28 | G98 | 244521_at | NM_173485 | — |
| — | G98 | 204304_s_at | NM_006017 | PROM1 Chr: 4p15.33 |
| — | G98 | 226863_at | NM_001077710 | FAM110C chr2p25.3 |

TABLE 7
Mouse probe-sets used for GSEA in Krivtsov dataset
Only most variant probe sets (sd >0.5) were retained
for GSEA
G98 Mouse Homologues

| Probesets | Gene.Title | Gene.Symbol |
|---|---|---|
| 1431475_a_at | homeo box A10 | HoxA10 |
| 1427433_s_at | homeo box A3 | HoxA3 |
| 1448926_at | homeo box A5 | HoxA5 |
| 1452421_at | homeo box A3 | HoxA3 |
| 1455626_at | homeo box A9 | HoxA9 |
| 1421579_at | homeo box A9 | HoxA9 |
| 1427454_at | homeo box C6 | HoxC6 |
| 1418879_at | RIKEN cDNA 9030611O19 gene | 9030611O19Rik |
| 1418895_at | src family associated | Scap2 |
| | phosphoprotein 2 | |
| 1419700_a_at | prominin 1 | Prom1 |
| 1449252_at | RIKEN cDNA 9030611O19 gene | 9030611O19Rik |
| 1450209_at | homeo box D4 | HoxD4 |

TABLE 9
Human probe-sets used for GSEA in Ross dataset
Only most variant probe sets (sd >0.5) were retained for GSEA
G98 U133A Corresponding Probesets to U133Plus2.0

| Probesets | Gene.Title | Gene.Symbol |
|---|---|---|
| 214651_s_at | homeobox A9 | HOXA9 |
| 213150_at | homeobox A10 | HOXA10 |
| 209905_at | homeobox A9 | HOXA9 |
| 213844_at | homeobox A5 | HOXA5 |
| 204362_at | src family associated phosphoprotein 2 | SCAP2 |
| 204304_s_at | prominin 1 | PROM1 |

TABLE 10
Gene Cluster G7

| | | RefSeq. | Chromosomal | | |
|---|---|---|---|---|---|
| Probe Id | Gene Symbol | Transcript ID | Location | Comments | Ref |
| 1556499_s_at | COL1A1 | NM_000088 | chr17q21.33 | Tumour endothelium marker | ¹¹ |
| 200700_s_at | KDELR2 | NM_001100603 | chr7p22.1 | | |
| | | /// NM_006854 | | | |
| 200771_at | LAMC1 | NM_002293 | chr1q31 | Basement membrane | |
| 201438_at | COL6A3 | NM_004369 /// | chr2q37 | Tumor endothelium marker | ¹¹ |
| | | NM_057164 /// | | | |
| | | NM_057165 /// | | | |
| | | NM_057166 /// | | | |
| | | NM_057167 | | | |
| 201505_at | LAMB1 | NM_002291 | chr7q22 | Basement membrane | |
| 201744_s_at | LUM | NM_002345 | chr12q21.3-q22 | Angiogenesis | ¹² |
| 201852_x_at | COL3A1 | NM_000090 | chr2q31 | Tumor endothelium marker | ¹¹ |
| 202007_at | NID1 | NM_002508 | chr1q43 | Tumor endothelium marker | ¹¹ |
| 202112_at | VWF | NM_000552 | chr12p13.3 | Endothelial marker | |
| 202202_s_at | LAMA4 | NM_001105206 | chr6q21 | Basement membrane | |
| | | /// | | | |
| | | NM_001105207 | | | |
| | | /// | | | |
| | | NM_001105208 | | | |
| | | /// | | | |
| | | NM_001105209 | | | |
| | | /// NM_002290 | | | |
| 202291_s_at | MGP | NM_000900 | chr12p13.1-p12.3 | Endothelial marker | ¹¹ |
| 202310_s_at | COL1A1 | NM_000088 | chr17q21.33 | Tumor endothelium marker | ¹¹ |
| 202375_at | SEC24D | NM_014822 | chr4q26 | | |
| 202403_s_at | COL1A2 | NM_000089 | chr7q22.1 | Tumor endothelium marker | ¹¹ |
| 202404_s_at | COL1A2 | NM_000089 | chr7q22.1 | Tumor endothelium marker | ¹¹ |
| 202465_at | PCOLCE | NM_002593 | chr7q22 | | |
| 202709_at | FMOD | NM_002023 | chr1q32 | | |
| 202766_s_at | FBN1 | NM_000138 | chr15q21.1 | Endothelial marker | |
| 202878_s_at | CD93 | NM_012072 | chr20p11.21 | | |
| 202952_s_at | ADAM12 | NM_003474 /// | chr10q26.3 | Cell adhesion | |
| | | NM_021641 | | | |
| 202998_s_at | LOXL2 | NM_002318 /// | chr8p21.3-p21.2 | | |
| | | NM_004901 | | | |
| 203325_s_at | COL5A1 | NM_000093 | chr9q34.2-q34.3 | | |
| 203851_at | IGFBP6 | NM_002178 | chr12q13 | Angiogenesis | |
| 204017_at | KDELR3 | NM_006855 /// | chr22q13.1 | | |
| | | NM_016657 | | | |
| 204083_s_at | TPM2 | NM_003289 /// | chr9p13.2-p13.1 | | |
| | | NM_213674 | | | |
| 204114_at | NID2 | NM_007361 | chr14q21-q22 | Tumor endothelium marker | ¹¹ |
| 204464_s_at | EDNRA | NM_001957 | chr4q31.23 | Patterning of blood vessels | GO |
| 204677_at | CDH5 | NM_001795 | chr16q22.1 | Endothelial marker | |
| 204682_at | LTBP2 | NM_000428 | chr14q24 | | |
| 204844_at | ENPEP | NM_001977 | chr4q25 | Angiogenesis | ¹³ |
| 205499_at | SRPX2 | NM_014467 | chrXq21.33-q23 | | |
| 205572_at | ANGPT2 | NM_001118887 | chr8p23.1 | Angiogenesis | |
| | | /// | | | |
| | | NM_001118888 | | | |
| | | /// NM_001147 | | | |
| 207714_s_at | SERPINH1 | NM_001235 | chr11q13.5 | | |
| 208690_s_at | PDLIM1 | NM_020992 | chr10q22-q26.3 | | |
| 209156_s_at | COL6A2 | NM_001849 /// | chr21q22.3 | Endothelial marker | |
| | | NM_058174 /// | | | |
| | | NM_058175 | | | |
| 209596_at | MXRA5 | NM_015419 | chrXp22.33 | | |
| 210495_x_at | FN1 | NM_002026 /// | chr2q34 | Basement membrane | |
| | | NM_054034 /// | | | |
| | | NM_212474 /// | | | |
| | | NM_212475 /// | | | |
| | | NM_212476 /// | | | |
| | | NM_212478 /// | | | |
| | | NM_212482 | | | |
| 211148_s_at | ANGPT2 | NM_001118887 | chr8p23.1 | Angiogenesis | |
| | | /// | | | |
| | | NM_001118888 | | | |
| | | /// NM_001147 | | | |
| 211161_s_at | COL3A1 | NM_000090 | chr2q31 | Tumor endothelium marker | ¹¹ |
| 211343_s_at | COL13A1 | NM_005203 /// | chr10q22 | | |
| | | NM_080798 /// | | | |
| | | NM_080799 /// | | | |
| | | NM_080800 /// | | | |
| | | NM_080801 /// | | | |
| | | NM_080802 /// | | | |
| | | NM_080803 /// | | | |
| | | NM_080804 /// | | | |
| | | NM_080805 /// | | | |
| | | NM_080806 /// | | | |
| | | NM_080807 /// | | | |
| | | NM_080808 /// | | | |
| | | NM_080809 /// | | | |
| | | NM_080810 /// | | | |
| | | NM_080811 /// | | | |
| | | NM_080812 /// | | | |
| | | NM_080813 /// | | | |
| | | NM_080814 /// | | | |
| | | NM_{—} | | | |
| 211651_s_at | LAMB1 | NM_002291 | chr7q22 | Basement membrane | |
| 211719_x_at | FN1 | NM_002026 /// | chr2q34 | Basement membrane | |
| | | NM_054034 /// | | | |
| | | NM_212474 /// | | | |
| | | NM_212475 /// | | | |
| | | NM_212476 /// | | | |
| | | NM_212478 /// | | | |
| | | NM_212482 | | | |
| 211964_at | COL4A2 | NM_001846 | chr13q34 | Glioma endothelial marker | ¹⁴ |
| 211966_at | COL4A2 | NM_001846 | chr13q34 | Glioma endothelial marker | ¹⁴ |
| 211980_at | COL4A1 | NM_001845 | chr13q34 | Glioma endothelial marker | ¹⁴ |
| 211981_at | COL4A1 | NM_001845 | chr13q34 | Glioma endothelial marker | ¹⁴ |
| 212298_at | NRP1 | NM_001024628 | chr10p12 | Angiogenesis | ¹⁵ |
| | | /// | | | |
| | | NM_001024629 | | | |
| | | /// NM_003873 | | | |
| 212364_at | MYO1B | NM_012223 | chr2q12-q34 | | |
| 212464_s_at | FN1 | NM_002026 /// | chr2q34 | Basement membrane | |
| | | NM_054034 /// | | | |
| | | NM_212474 /// | | | |
| | | NM_212475 /// | | | |
| | | NM_212476 /// | | | |
| | | NM_212478 /// | | | |
| | | NM_212482 | | | |
| 212488_at | COL5A1 | NM_000093 | chr9q34.2-q34.3 | | |
| 212489_at | COL5A1 | NM_000093 | chr9q34.2-q34.3 | | |
| 213125_at | OLFML2B | NM_015441 | chr1q23.3 | | |
| 213139_at | SNAI2 | NM_003068 | chr8q11 | | |
| 213790_at | — | — | — | | |
| 214081_at | PLXDC1 | NM_020405 | chr17q21.1 | | |
| 215076_s_at | COL3A1 | NM_000090 | chr2q31 | Tumor endothelium marker | ¹¹ |
| 216442_x_at | FN1 | NM_002026 /// | chr2q34 | Basement membrane | |
| | | NM_054034 /// | | | |
| | | NM_212474 /// | | | |
| | | NM_212475 /// | | | |
| | | NM_212476 /// | | | |
| | | NM_212478 /// | | | |
| | | NM_212482 | | | |
| 218729_at | LXN | NM_020169 | chr3q25.32 | | |
| 219134_at | ELTD1 | NM_022159 | chr1p33-p32 | | |
| 219773_at | NOX4 | NM_016931 | chr11q14.2-q21 | oxygen sensor activity | ¹⁶ |
| 221729_at | COL5A2 | NM_000393 | chr2q14-q32 | | |
| 221730_at | COL5A2 | NM_000393 | chr2q14-q32 | | |
| 224833_at | ETS1 | NM_005238 | chr11q23.3 | Angiogenesis | ¹⁷ |
| 225681_at | CTHRC1 | NM_138455 | chr8q22.3 | Vascular remodeling | ¹⁸ |
| 225799_at | MGC4677 /// | XR_039885 /// | chr2p11.2 /// chr2q13 | | |
| | LOC541471 | XR_039886 /// | | | |
| | | XR_042051 /// | | | |
| | | XR_042052 | | | |
| 226311_at | — | — | — | | |
| 226731_at | PELO | NM_015946 | chr5q11.2 | | |
| 226777_at | — | — | — | | |
| 226804_at | FAM20A | NM_017565 | chr17q24.2 | | |
| 227628_at | LOC493869 | NM_001008397 | chr5q11.2 | | |
| 228776_at | GJA7 | NM_001080383 | chr17q21.31 | | |
| | | /// NM_005497 | | | |
| 229218_at | COL1A2 | NM_000089 | chr7q22.1 | Tumor endothelium marker | ¹¹ |
| 230061_at | TM4SF18 | NM_138786 | chr3q25.1 | | |
| 232458_at | COL3A1 | NM_000090 | chr2q31 | Tumor endothelium marker | ¹¹ |
| 236034_at | — | NM_001118887 | — | | |
| | | /// | | | |
| | | NM_001118888 | | | |
| | | /// NM_001147 | | | |
| 237261_at | — | NM_001118887 | — | | |
| | | /// | | | |
| | | NM_001118888 | | | |
| | | /// NM_001147 | | | |
| 241981_at | FAM20A | NM_017565 | chr17q24.2 | | |

TABLE 11
Gene Cluster G13

| | Gene | | Chromosomal |
|---|---|---|---|
| Probe Id | Symbol | RefSeq.Transcript.ID | Location |
| 1555385_at | B4GALNT1 | NM_001478 | chr12q13.3 |
| 1568706_s_at | AVIL | NM_006576 | chr12q14.1 |
| | | NM_001017956 /// | |
| | | NM_001017957 /// | |
| | | NM_001017958 /// | |
| 200714_x_at | OS9 | NM_006812 | chr12q13 |
| 201131_s_at | CDH1 | NM_004360 | chr16q22.1 |
| 202246_s_at | CDK4 | NM_000075 | chr12q14 |
| 203226_s_at | TSPAN31 | NM_005981 | chr12q13.3 |
| 203227_s_at | TSPAN31 | NM_005981 | chr12q13.3 |
| | | NM_005371 /// | |
| 204027_s_at | METTL1 | NM_023033 | chr12q13 |
| | | NM_002392 /// | |
| | | NM_006878 /// | |
| | | NM_006879 /// | |
| | | NM_006881 /// | |
| 205386_s_at | MDM2 | NM_006882 | chr12q14.3-q15 |
| 205539_at | AVIL | NM_006576 | chr12q14.1 |
| 205676_at | CYP27B1 | NM_000785 | chr12q13.1-q13.3 |
| | | NM_001005502 /// | |
| | | NM_001874 /// | |
| 206100_at | CPM | NM_198320 | chr12q14.3 |
| 206435_at | B4GALNT1 | NM_001478 | chr12q13.3 |
| | | NM_002392 /// | |
| | | NM_006878 /// | |
| | | NM_006879 /// | |
| | | NM_006881 /// | |
| 211832_s_at | MDM2 | NM_006882 | chr12q14.3-q15 |
| 212656_at | TSFM | NM_005726 | chr12q13-q14 |
| 213861_s_at | FAM119B | NM_015433 /// | chr12q14.1 |
| | | NM_206914 | |
| 214331_at | TSFM | NM_005726 | chr12q13-q14 |
| 214332_s_at | TSFM | NM_005726 | chr12q13-q14 |
| 214951_at | SLC26A10 | NM_133489 | chr12q13 |
| | | NM_001017956 /// | |
| | | NM_001017957 /// | |
| | | NM_001017958 /// | |
| 215399_s_at | OS9 | NM_006812 | chr12q13 |
| | | NM_002392 /// | |
| | | NM_006878 /// | |
| | | NM_006879 /// | |
| | | NM_006881 /// | |
| 217373_x_at | MDM2 | NM_006882 | chr12q14.3-q15 |
| 217542_at | CPM | — | chr12q14.3 |
| 218768_at | NUP107 | NM_020401 | chr12q15 |
| 224489_at | KIAA1267 | NM_015443 | chr17q21.31 |
| 225160_x_at | MGC5370 | — | chr12q14.3 |
| 226454_at | MARCH9 | NM_138396 | chr12q14.1 |
| 226546_at | — | — | — |
| 227678_at | XRCC6BP1 | NM_033276 | chr12q14.1 |
| 229711_s_at | MGC5370 | — | chr12q14.3 |
| 229917_at | — | — | — |
| 230001_at | MARCH9 | NM_138396 | chr12q14.1 |
| | | NM_001005502 /// | |
| | | NM_001874 /// | |
| 235019_at | CPM | NM_198320 | chr12q14.3 |
| | | NM_001005502 /// | |
| | | NM_001874 /// | |
| 235706_at | CPM | NM_198320 | chr12q14.3 |
| 235721_at | DTX3 | NM_178502 | chr12q13.3 |
| 238733_at | CPM | — | chr12q14.3 |
| 238999_at | AVIL | — | chr12q14.1 |
| | | NM_001005502 /// | |
| 241765_at | CPM | NM_001874 /// | chr12q14.3 |
| | | NM_198320 | |
| | | NM_001102450 /// | |
| 244675_at | RGS8 | NM_033345 | chr1q25 |

TABLE 12
Gene Cluster G14

| | | | Chromosomal | | |
|---|---|---|---|---|---|
| Probe Id | Gene Symbol | RefSeq. Transcript. ID | Location | Comments | Ref. |
| 203180_at | ALDH1A3 | NM_000693 | chr15q26.3 | Differentiation of stem cells | ¹⁹ |
| 203434_s_at | MME | NM_000902 /// | chr3q25.1-q25.2 | | |
| | | NM_007287 /// | | | |
| | | NM_007288 /// | | | |
| | | NM_007289 | | | |
| 204776_at | THBS4 | NM_003248 | chr5q13 | | |
| 205430_at | BMP5 | NM_021073 | chr6p12.1 | Differentiation of stem cells | ²⁰ |
| 205431_s_at | BMP5 | NM_021073 | chr6p12.1 | Differentiation of stem cells | ²⁰ |
| 205619_s_at | MEOX1 | NM_001040002 /// | chr17q21 | Regulator of BMPs | ²¹ |
| | | NM_004527 /// | | | |
| | | NM_013999 | | | |
| 205713_s_at | COMP | NM_000095 | chr19p13.1 | | |
| 205848_at | GAS2 | NM_005256 /// | chr11p14.3-p15.2 | Apoptosis | |
| | | NM_177553 | | | |
| 212414_s_at | SEPT6 /// N-PAC | NM_015129 /// | chrXq24 /// chr16p13.3 | | |
| | | NM_032569 /// | | | |
| | | NM_145799 /// | | | |
| | | NM_145800 /// | | | |
| | | NM_145802 | | | |
| 213456_at | SOSTDC1 | NM_015464 | chr7p21.1 | Regulator of BMPs | ²² |
| 217525_at | OLFML1 | NM_198474 | chr11p15.4 | | |
| 218499_at | RP6-213H19.1 | NM_001042452 /// | chrXq26.2 | | |
| | | NM_001042453 /// | | | |
| | | NM_016542 | | | |
| 219837_s_at | CYTL1 | NM_018659 | chr4p16-p15 | | |
| 220014_at | PRR16 | NM_016644 | chr5q23.1 | Mesenchymal stem cell marker | ²³ |
| 220065_at | TNMD | NM_022144 | chrXq21.33-q23 | | |
| 226834_at | — | — | — | | |
| 226930_at | FNDC1 | NM_032532 | chr6q25 | | |
| 227070_at | GLT8D2 | NM_031302 | chr12q | | |
| 227850_x_at | CDC42EP5 | NM_145057 | chr19q13.42 | | |
| 229839_at | SCARA5 | NM_173833 | chr8p21.1 | | |
| 231766_s_at | COL12A1 | NM_004370 /// | chr6q12-q13 | | |
| | | NM_080645 | | | |
| 232090_at | DNM3 | XM_001715447 /// | chr1q24.3 | | |
| | | XM_001718852 /// | | | |
| | | XM_001718912 | | | |
| 235944_at | HMCN1 | NM_031935 | chr1q25.3-q31.1 | | |
| 236035_at | — | — | — | | |
| 239468_at | MKX | NM_173576 | chr10p12.1 | Homeobox gene | ²⁴ |
| 244885_at | — | — | — | | |

TABLE 13
Gene Cluster G18

| | Gene | | Chromosomal |
|---|---|---|---|
| Probe Id | Symbol | RefSeq.Transcript.ID | Location |
| 202800_at | SLC1A3 | NM_004172 | chr5p13 |
| 203723_at | ITPKB | NM_002221 | chr1q42.13 |
| 204041_at | MAOB | NM_000898 | chrXp11.23 |
| 204363_at | F3 | NM_001993 | chr1p22-p21 |
| 205363_at | BBOX1 | NM_003986 | chr11p14.2 |
| | | NM_001390 /// | |
| | | NM_001391 /// | |
| | | NM_001392 /// | |
| | | NM_032975 /// | |
| | | NM_032978 /// | |
| | | NM_032979 /// | |
| | | NM_032980 /// | |
| 205741_s_at | DTNA | NM_032981 | chr18q12 |
| 206022_at | NDP | NM_000266 | chrXp11.4 |
| 207443_at | NR2E1 | NM_003269 | chr6q21 |
| 207455_at | P2RY1 | NM_002563 | chr3q25.2 |
| 209301_at | CA2 | NM_000067 | chr8q22 |
| 209921_at | SLC7A11 | NM_014331 | chr4q28-q32 |
| | | NM_001650 /// | |
| 210066_s_at | AQP4 | NM_004028 | chr18q11.2-q12.1 |
| | | NM_001650 /// | |
| 210067_at | AQP4 | NM_004028 | chr18q11.2-q12.1 |
| | | NM_001650 /// | |
| 210068_s_at | AQP4 | NM_004028 | chr18q11.2-q12.1 |
| | | NM_001650 /// | |
| 210906_x_at | AQP4 | NM_004028 | chr18q11.2-q12.1 |
| | | NM_015166 /// | |
| 213395_at | MLC1 | NM_139202 | chr22q13.33 |
| | | NM_015230 /// | |
| 213618_at | CENTD1 | NM_139182 | chr4p14 |
| 217678_at | SLC7A11 | NM_014331 | chr4q28-q32 |
| 223605_at | SLC25A18 | NM_031481 | chr22q11.2 |
| 226189_at | ITGB8 | NM_002214 | chr7p15.3 |
| | | NM_001650 /// | |
| 226228_at | AQP4 | NM_004028 | chr18q11.2-q12.1 |
| | | NM_001390 /// | |
| | | NM_001391 /// | |
| | | NM_001392 /// | |
| | | NM_032975 /// | |
| | | NM_032978 /// | |
| | | NM_032979 /// | |
| | | NM_032980 /// | |
| 227084_at | DTNA | NM_032981 | chr18q12 |
| 231925_at | P2RY1 | — | chr3q25.2 |
| | | NM_000280 /// | |
| 235795_at | PAX6 | NM_001604 | chr11p13 |
| 238003_at | FLJ25530 | NM_152722 | chr11q24.2 |

TABLE 14
Gene Cluster G24

| | | RefSeq. | Chromosomal | | |
|---|---|---|---|---|---|
| Probe Id | Gene Symbol | Transcript ID | Location | Comments | Ref |
| 1552316_a_at | GIMAP1 | NM_130759 | chr7q36.1 | | |
| 1552365_at | SCIN | NM_001112706 | chr7p21.3 | | |
| | | /// NM_033128 | | | |
| 1552367_a_at | SCIN | NM_001112706 | chr7p21.3 | | |
| | | /// NM_033128 | | | |
| 1552386_at | C5orf29 | NM_152687 | chr5q11.2 | | |
| 1552807_a_at | SIGLEC10 | NM_033130 | chr19q13.3 | | |
| 1554899_s_at | FCER1G | NM_004106 | chr1q23 | M2 Marker | ²⁵ |
| 1555349_a_at | ITGB2 | NM_000211 | chr21q22.3 | Macrophage | |
| | | | | markers | |
| 1555728_a_at | MS4A4A | NM_024021 /// | chr11q12 | | |
| | | NM_148975 | | | |
| 201137_s_at | HLA-DPB1 | NM_002121 | chr6p21.3 | | ²⁵ |
| 201422_at | IFI30 | NM_005027 /// | chr19p13.1 | | |
| | | NM_006332 | | | |
| 201487_at | CTSC | NM_001114173 | chr11q14.1-q14.3 | M2 Marker | ²⁶ |
| | | /// NM_001814 /// | | | |
| | | NM_148170 | | | |
| 201631_s_at | IER3 | NM_003897 | chr6p21.3 | | |
| 201721_s_at | LAPTM5 | NM_006762 | chr1p34 | | |
| 201743_at | CD14 | NM_000591 /// | chr5q22-q32\|5q31.1 | Innate immunity | |
| | | NM_001040021 | | | |
| 202546_at | VAMP8 | NM_003761 | chr2p12-p11. | | |
| 202803_s_at | ITGB2 | NM_000211 | chr21q22.3 | | |
| 202833_s_at | SERPINA1 | NM_000295 /// | chr14q32.1 | | |
| | | NM_001002235 | | | |
| | | /// | | | |
| | | NM_001002236 | | | |
| 202901_x_at | CTSS | NM_004079 | chr1q21 | M2 Marker | ²⁶ |
| 202902_s_at | CTSS | NM_004079 | chr1q21 | M2 Marker | ²⁶ |
| 202917_s_at | S100A8 | NM_002964 | chr1q21 | | |
| 202953_at | C1QB | NM_000491 | chr1p36.12 | M2 Marker | ²⁷ |
| 202957_at | HCLS1 | NM_005335 | chr3q13 | | |
| 203104_at | CSF1R | NM_005211 | chr5q33-q35 | | |
| 203290_at | HLA-DQA1 | NM_002122 /// | chr6p21.3 | | |
| | | XM_001722240 | | | |
| | | /// | | | |
| | | XM_001723439 | | | |
| 203305_at | F13A1 | NM_000129 | chr6p25.3-p24.3 | M2 Marker | ²⁶ |
| 203416_at | CD53 | NM_000560 /// | chr1p13 | Macrophage | |
| | | NM_001040033 | | markers | |
| 203535_at | S100A9 | NM_002965 | chr1q21 | | |
| 203561_at | FCGR2A | NM_021642 | chr1q23 | | |
| 203645_s_at | CD163 | NM_004244 /// | chr12p13.3 | M2 Marker | ²⁸ |
| | | NM_203416 | | | |
| 203665_at | HMOX1 | NM_002133 | chr22q12\|22q13.1 | | |
| 204006_s_at | FCGR3A /// | NM_000569 /// | chr1q23 | | |
| | FCGR3B | NM_000570 | | | |
| 204007_at | FCGR3B | NM_000570 | chr1q23 | | |
| 204122_at | TYROBP | | chr19q13.1 | Innate immunity | |
| 204150_at | STAB1 | NM_015136 | chr3p21.1 | M2 Marker | ²⁹ |
| 204174_at | ALOX5AP | NM_001629 | chr13q12 | M2 Marker | ²⁵ |
| 204232_at | FCER1G | NM_004106 | chr1q23 | M2 Marker | ²⁵ |
| 204416_x_at | APOC1 | NM_001645 | chr19q13.2 | | |
| 204430_s_at | SLC2A5 | NM_003039 | chr1p36.2 | | |
| 204438_at | MRC1 /// MRC1L1 | NM_001009567 | chr10p12.33 | M2 Marker | ³⁰ |
| | | /// NM_002438 | | | |
| 204446_s_at | ALOX5 | NM_000698 | chr10q11.2 | M2 Marker | 25 |
| 204563_at | SELL | NM_000655 | chr1q23-q25 | | |
| 204670_x_at | HLA-DRB1 | NM_001023561 | chr6p21.3 | | 25 |
| | | /// NM_002123 /// | | | |
| | | NM_002124 /// | | | |
| | | NM_002125 /// | | | |
| | | NM_002934 /// | | | |
| | | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 204787_at | VSIG4 | NM_001100431 | chrXq12-q13.3 | | |
| | | /// NM_007268 | | | |
| 204834_at | FGL2 | NM_006682 | chr7q11.23 | | |
| 204959_at | MNDA | NM_002432 | chr1q22 | | |
| 204971_at | CSTA | NM_005213 | chr3q21 | | |
| 205027_s_at | MAP3K8 | NM_005204 | chr10p11.23 | | |
| 205119_s_at | FPR1 | NM_002029 | chr19q13.4 | | |
| 205681_at | BCL2A1 | NM_001114735 | chr15q24.3 | | |
| | | /// NM_004049 | | | |
| 205786_s_at | ITGAM | NM_000632 | chr16p11.2 | | |
| 205890_s_at | UBD | NM_001470 /// | chr6p21.3 | | |
| | | NM_006398 /// | | | |
| | | NM_021903 /// | | | |
| | | NM_021904 /// | | | |
| | | NM_021905 | | | |
| 205898_at | CX3CR1 | NM_001337 | chr3p21\|3p21.3 | | |
| 205997_at | ADAM28 | NM_014265 /// | chr8p21.2 | | |
| | | NM_021777 | | | |
| 206111_at | HLA-DQB1 /// HLA- | NM_001023561 | chr14q24-q31 | | |
| | DQB2 /// HLA- | /// NM_002123 /// | | | |
| | DRB1 /// HLA-DRB2 | NM_002124 /// | | | |
| | /// HLA-DRB3 /// | NM_002125 /// | | | |
| | HLA-DRB4 /// HLA- | NM_002934 /// | | | |
| | DRB5 | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 206420_at | IGSF6 | NM_005849 | chr16p12-p13 | | |
| 206584_at | LY96 | NM_015364 | chr8q21.11 | | |
| 207655_s_at | BLNK | NM_001114094 | chr10q23.2-q23.33 | | |
| | | /// NM_013314 | | | |
| 208018_s_at | HCK | NM_002110 | chr20q11-q12 | | |
| 208146_s_at | CPVL | NM_019029 /// | chr7p15-p14 | | |
| | | NM_031311 | | | |
| 208306_x_at | HLA-DRB1 | NM_002124 | chr6p21.3 | | 25 |
| 208894_at | HLA-DRA | NM_019111 | chr6p21.3 | | 25 |
| 208944_at | TGFBR2 | NM_001024847 | chr3p22 | | 31 |
| | | /// NM_003242 | | | |
| 209312_x_at | HLA-DRB1 | NM_001023561 | chr6p21.3 | | 25 |
| | | /// NM_002123 /// | | | |
| | | NM_002124 /// | | | |
| | | NM_002125 /// | | | |
| | | NM_002934 /// | | | |
| | | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 209619_at | CD74 | NM_001025158 | chr5q32 | | |
| | | /// | | | |
| | | NM_001025159 | | | |
| | | /// NM_004355 | | | |
| 209823_x_at | HLA-DQB1 | NM_002123 /// | chr6p21.3 | | 25 |
| | | XM_001722253 | | | |
| | | /// | | | |
| | | XM_001723447 | | | |
| 210176_at | TLR1 | NM_003263 | chr4p14 | Innate immunity | |
| 210314_x_at | TNFSF13 /// | NM_003808 | chr17p13.1 | | |
| | TNFSF12-TNFSF13 | /// | | | |
| | | NM_172087 | | | |
| | | /// | | | |
| | | NM_172088 | | | |
| 210982_s_at | HLA-DRA | NM_019111 | chr6p21.3 | | 25 |
| 211429_s_at | SERPINA1 | NM_000295 /// | chr14q32.1 | | |
| | | NM_001002235 | | | |
| | | /// | | | |
| | | NM_001002236 | | | |
| 211654_x_at | HLA-DQB1 /// | NM_002123 /// | chr6p21.3 | | |
| | LOC650557 | XM_001722253 | | | |
| | | /// | | | |
| | | XM_001723447 | | | |
| 211656_x_at | HLA-DQB1 | NM_002123 /// | chr6p21.3 | | 25 |
| | | XM_001722253 | | | |
| | | /// | | | |
| | | XM_001723447 | | | |
| 211742_s_at | EVI2B | NM_006495 | chr17q11.2 | | |
| 211990_at | HLA-DPA1 | NM_033554 | chr6p21.3 | | 25 |
| 211991_s_at | HLA-DPA1 | NM_033554 | chr6p21.3 | | 25 |
| 212543_at | AIM1 | NM_001624 | chr6q21 | | |
| 212588_at | PTPRC | NM_002838 /// | chr1q31-q32 | Macrophage | |
| | | NM_080921 /// | | markers | |
| | | NM_080922 /// | | | |
| | | NM_080923 | | | |
| 212671_s_at | HLA-DQA1 /// HLA- | NM_002122 /// | chr6p21.3 | | 25 |
| | DQA2 | NM_020056 /// | | | |
| | | XM_001722240 | | | |
| | | /// | | | |
| | | XM_001723439 | | | |
| 212998_x_at | HLA-DQB1 | NM_001023561 | chr6p21.3 | | 25 |
| | | /// NM_002123 /// | | | |
| | | NM_002124 /// | | | |
| | | NM_002125 /// | | | |
| | | NM_002934 /// | | | |
| | | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 213537_at | HLA-DPA1 | NM_033554 | chr6p21.3 | | 25 |
| 213566_at | RNASE6 | NM_005615 | chr14q11.2 | | |
| 214467_at | GPR65 | NM_003608 | chr14q31-q32.1 | | |
| 214511_x_at | FCGR1A /// | NM_001004340 | chr1q21.2-q21.3 | | |
| | | /// | /// | | |
| | | NM_001017986 | chr1p11.2 | | |
| 214770_at | MSR1 | NM_002445 /// | chr8p22 | M2 Marker | 25 |
| | | NM_138715 /// | | | |
| | | NM_138716 | | | |
| 215049_x_at | CD163 | NM_004244 /// | chr12p13.3 | M2 Marker | 28 |
| | | NM_203416 | | | |
| 215193_x_at | HLA-DRB1 | NM_001023561 | chr6p21.3 | | ²⁵ |
| | | /// NM_002123 /// | | | |
| | | NM_002124 /// | | | |
| | | NM_002125 /// | | | |
| | | NM_002934 /// | | | |
| | | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 216233_at | CD163 | NM_004244 /// | chr12p13.3 | M2 Marker | ²⁸ |
| | | NM_203416 | | | |
| 216950_s_at | FCGR1A | NM_000566 | chr1q21.2-q21.3 | | |
| 217388_s_at | KYNU | NM_001032998 | chr2q22.2 | | |
| | | /// NM_003937 | | | |
| 217478_s_at | HLA-DMA | NM_006120 | chr6p21.3 | | ²⁵ |
| 217767_at | C3 /// LOC653879 | NM_000064 | chr19p13.3-p13.2 | Innate immunity | |
| 217983_s_at | RNASET2 | NM_003730 | chr6q27 | | |
| 218232_at | C1QA | NM_015991 | chr1p36.12 | M2 Marker | ²⁷ |
| 218854_at | SART2 | NM_001080976 | chr6q22 | | |
| | | /// NM_013352 | | | |
| 219386_s_at | SLAMF8 | NM_020125 | chr1q23.2 | | |
| 219607_s_at | MS4A4A | NM_024021 /// | chr11q12 | | |
| | | NM_148975 | | | |
| 219666_at | MS4A6A | NM_022349 /// | chr11q12.1 | | |
| | | NM_152851 /// | | | |
| | | NM_152852 | | | |
| 219890_at | CLEC5A | NM_013252 | chr7q33 | | |
| 220005_at | P2RY13 | NM_023914 /// | chr3q24 | | |
| | | NM_176894 | | | |
| 220146_at | TLR7 | NM_016562 | chrXp22.3 | Innate immunity | |
| 220330_s_at | SAMSN1 | NM_022136 | chr21q11 | | |
| 220491_at | HAMP | NM_021175 | chr19q13.1 | Innate immunity | |
| 220532_s_at | LR8 | NM_001101311 | chr7q36.1 | | |
| | | /// | | | |
| | | NM_001101312 | | | |
| | | /// | | | |
| | | NM_001101313 | | | |
| | | /// | | | |
| | | NM_001101314 | | | |
| | | /// NM_014020 | | | |
| 221210_s_at | NPL | NM_030769 | chr1q25 | | |
| 221491_x_at | HLA-DRB1 /// HLA- | NM_001023561 | chr6p21.3 | | ²⁵ |
| | DRB3 /// HLA-DRB4 | /// NM_002123 /// | | | |
| | /// H LA-DRB5 | NM_002124 /// | | | |
| | | NM_002125 /// | | | |
| | | NM_002934 /// | | | |
| | | NM_021983 /// | | | |
| | | NM_022555 /// | | | |
| | | NR_003937 /// | | | |
| | | XM_001124749 | | | |
| | | /// | | | |
| | | XM_001713857 | | | |
| | | /// | | | |
| | | XM_001713867 | | | |
| | | /// | | | |
| | | XM_001714067 | | | |
| | | /// | | | |
| | | XM_001714074 | | | |
| | | /// | | | |
| | | XM_001718754 | | | |
| | | /// | | | |
| | | XM_001719473 | | | |
| | | /// | | | |
| | | XM_001720834 | | | |
| | | /// XM_0 | | | |
| 221698_s_at | CLEC7A | NM_022570 /// | chr12p13.2 | M2 Marker | ³² |
| | | NM_197947 /// | | | |
| | | NM_197948 /// | | | |
| | | NM_197949 /// | | | |
| | | NM_197950 /// | | | |
| | | NM_197954 | | | |
| 223280_x_at | MS4A6A | NM_022349 /// | chr11q12.1 | | |
| | | NM_152851 /// | | | |
| | | NM_152852 | | | |
| 223343_at | MS4A7 | NM_021201 /// | chr11q12 | | |
| | | NM_206938 /// | | | |
| | | NM_206939 /// | | | |
| | | NM_206940 | | | |
| 223620_at | GPR34 | NM_001097579 | chrXp11.4-p11.3 | | |
| | | /// NM_005300 | | | |
| 223660_at | ADORA3 | NM_000677 /// | chr1p13.2 | | |
| | | NM_001081976 | | | |
| | | /// NM_020683 | | | |
| 224356_x_at | MS4A6A | NM_022349 /// | chr11q12.1 | | |
| | | NM_152851 /// | | | |
| | | NM_152852 | | | |
| 225353_s_at | C1QC | NM_001114101 | chr1p36.11 | M2 Marker | ²⁷ |
| | | /// NM_172369 | | | |
| 225502_at | DOCK8 | NM_203447 | chr9p24.3 | | |
| 225646_at | CTSC | NM_001114173 | chr11q14.1-q14.3 | M2 Marker | ²⁶ |
| | | /// NM_001814 /// | | | |
| | | NM_148170 | | | |
| 225647_s_at | CTSC | NM_001114173 | chr11q14.1-q14.3 | M2 Marker | ²⁶ |
| | | /// NM_001814 /// | | | |
| | | NM_148170 | | | |
| 226068_at | SYK | NM_003177 | chr9q22 | | |
| 227265_at | FGL2 | NM_006682 | chr7q11.23 | | |
| 227266_s_at | FYB | NM_001465 /// | chr5p13.1 | | |
| | | NM_199335 | | | |
| 227346_at | ZNFN1A1 | NM_006060 | chr7p13-p11.1 | | |
| 227889_at | AYTL1 | NM_017839 | chr16q12.2 | | |
| 228532_at | C1orf162 | NM_174896 | chr1p13.2 | | |
| 229074_at | — | — | — | | |
| 229560_at | TLR8 | NM_138636 | chrXp22 | Innate immunity | |
| 229723_at | TAGAP | NM_054114 /// | chr6q25.3 | | |
| | | NM_138810 /// | | | |
| | | NM_152133 | | | |
| 229937_x_at | LILRB1 | NM_001081637 | chr19q13.4 | | |
| | | /// | | | |
| | | NM_001081638 | | | |
| | | /// | | | |
| | | NM_001081639 | | | |
| | | /// NM_006669 | | | |
| 230252_at | GPR92 | NM_020400 | chr12p13.31 | | |
| 230391_at | — | — | — | | |
| 230550_at | MS4A6A | NM_022349 /// | chr11q12.1 | | |
| | | NM_152851 /// | | | |
| | | NM_152852 | | | |
| 230925_at | APBB1IP | NM_019043 | chr10p12.1 | | |
| 232617_at | CTSS | NM_004079 | chr1q21 | M2 Marker | ²⁶ |
| 232843_s_at | DOCK8 | NM_203447 | chr9p24.3 | | |
| 234987_at | C20orf118 | — | chr20q11.23 | | |
| 236028_at | IBSP | NM_004967 | chr4q21-q25 | | |
| 244434_at | — | — | — | | |
| 38487_at | STAB1 | NM_015136 | chr3p21.1 | M2 Marker | |

TABLE 15
Gene Cluster G25

| | Gene | | Chromosomal |
|---|---|---|---|
| ProbeId | Symbol | RefSeq.Transcript.ID | Location |
| | | NM_005228 /// NM_201282 /// | |
| 201983_s_at | EGFR | NM_201283 /// NM_201284 | chr7p12 |
| | | NM_005228 /// NM_201282 /// | |
| 201984_s_at | EGFR | NM_201283 /// NM_201284 | chr7p12 |
| 203373_at | SOCS2 | NM_003877 | chr12q |
| 203484_at | SEC61G | NM_001012456 /// NM_014302 | chr7p11.2 |
| | | NM_005244 /// NM_172110 /// | |
| | | NM_172111 /// NM_172112 /// | |
| 209692_at | EYA2 | NM_172113 | chr20q13.1 |
| 210135_s_at | SHOX2 | NM_003030 /// NM_006884 | chr3q25-q26.1 |
| 224999_at | EGFR | — | chr7p12 |
| 228307_at | EMILIN3 | NM_052846 | chr20q11.2-q12 |
| 232120_at | EGFR | — | chr7p12 |
| | | NM_001031849 /// NM_001879 /// | |
| 232224_at | MASP1 | NM_139125 | chr3q27-q28 |
| 232539_at | — | — | — |
| 232541_at | EGFR | — | chr7p12 |
| 232882_at | FOXO1A | — | chr13q14.1 |
| 232925_at | EGFR | — | chr7p12 |
| 232935_at | LHFP | — | chr13q12 |
| 233025_at | PDZD2 | NM_178140 | chr5p13.3 |
| 233044_at | EGFR | — | chr7p12 |
| 243327_at | EGFR | — | chr7p12 |

### REFERENCE LIST

Abate-Shen, C. (2002). Deregulated homeobox gene expression in cancer: cause or consequence? Nat Rev Cancer 2, 777-785.
Abdel-Fattah, R., Xiao, A., Bomgardner, D., Pease, C. S., Lopes, M. B., and Hussaini, I. M. (2006). Differential expression of HOX genes in neoplastic and non-neoplastic human astrocytes. J Pathol 209, 15-24.
Bachoo, R. M., Maher, E. A., Ligon, K. L., Sharpless, N. E., Chan, S. S., You, M. J., Tang, Y., DeFrances, J., Stover, E., Weissleder, R., et al. (2002). Epidermal growth factor receptor and Ink4a/Arf: convergent mechanisms governing terminal differentiation and transformation along the neural stem cell to astrocyte axis. Cancer Cell 1, 269-277.
Bao, S., Wu, Q., McLendon, R. E., Hao, Y., Shi, Q., Hjelmeland, A. B., Dewhirst, M. W., Bigner, D. D., and Rich, J. N. (2006). Glioma stem cells promote radioresistance by preferential activation of the DNA damage response. Nature 444, 756-760.
Batchelor, T. T., Sorensen, A. G., di Tomaso, E., Zhang, W. T., Duda, D. G., Cohen, K. S., Kozak, K. R., Cahill, D. P., Chen, P. J., Zhu, M., et al. (2007). AZD2171, a pan-VEGF receptor tyrosine kinase inhibitor, normalizes tumor vasculature and alleviates edema in glioblastoma patients. Cancer Cell 11, 83-95.
Calabrese, C., Poppleton, H., Kocak, M., Hogg, T. L., Fuller, C., Hamner, B., Oh, E. Y., Gaber, M. W., Finklestein, D., Allen, M., et al. (2007). A perivascular niche for brain tumor stem cells. Cancer Cell 11, 69-82.
Chute, J. P., Muramoto, G. G., Whitesides, J., Colvin, M., Safi, R., Chao, N. J., and McDonnell, D. P. (2006). Inhibition of aldehyde dehydrogenase and retinoid signaling induces the expansion of human hematopoietic stem cells. Proc Natl Acad Sci USA 103, 11707-11712.
Clement, V., Sanchez, P., de Tribolet, N., Radovanovic, I., and Ruiz i Altaba, A. (2007). HEDGEHOG-GL11 signaling regulates human glioma growth, cancer stem cell self-renewal, and tumorigenicity. Curr Biol 17, 165-172.
Dorrance, A. M., Liu, S., Yuan, W., Becknell, B., Arnoczky, K. J., Guimond, M., Strout, M. P., Feng, L., Nakamura, T., Yu, L., et al. (2006). M11 partial tandem duplication induces aberrant Hox expression in vivo via specific epigenetic alterations. J Clin Invest 116, 2707-2716.
Freije, W. A., Castro-Vargas, F. E., Fang, Z., Horvath, S., Cloughesy, T., Liau, L. M., Mischel, P. S., and Nelson, S. F. (2004). Gene expression profiling of gliomas strongly predicts survival. Cancer Res 64, 6503-6510.
Getz, G., Levine, E., and Domany, E. (2000). Coupled two-way clustering analysis of gene microarray data. Proc Natl Acad Sci USA 97, 12079-12084.
Hegi, M. E., Diserens, A. C., Gorlia, T., Hamou, M. F., de Tribolet, N., Weller, M., Kros, J. M., Hainfellner, J. A., Mason, W. P., Mariani, L., et al. (2005). MGMT gene silencing and benefit from temozolomide in glioblastoma. New Engl J Med 352, 997-1003.
Herrlich, A., Leitch, V., and King, L. S. (2004). Role of proneuregulin 1 cleavage and human epidermal growth factor receptor activation in hypertonic aquaporin induction. Proc Natl Acad Sci USA 101, 15799-15804.
Kjellman, C., Olofsson, S. P., Hansson, 0., Von Schantz, T., Lindvall, M., Nilsson, I., Salford, L. G., Sjogren, H. O., and Widegren, B. (2000). Expression of TGF-beta isoforms, TGF-beta receptors, and SMAD molecules at different stages of human glioma. Int J Cancer 89, 251-258.
Krivtsov, A. V., Twomey, D., Feng, Z., Stubbs, M. C., Wang, Y., Faber, J., Levine, J. E., Wang, J., Hahn, W. C., Gilliland, D. G., et al. (2006). Transformation from committed progenitor to leukaemia stem cell initiated by MLL-AF9. Nature 442, 818-822.
Madden, S. L., Cook, B. P., Nacht, M., Weber, W. D., Callahan, M. R., Jiang, Y., Dufault, M. R., Zhang, X., Zhang, W., Walter-Yohrling, J., et al. (2004). Vascular gene expression in normeoplastic and malignant brain. Am J Pathol 165, 601-608.
Manley, G. T., Fujimura, M., Ma, T., Noshita, N., Filiz, F., Bollen, A. W., Chan, P., and Verkman, A. S. (2000). Aquaporin-4 deletion in mice reduces brain edema after acute water intoxication and ischemic stroke. Nat Med 6, 159-163.
Mantovani, A., Sozzani, S., Locati, M., Allavena, P., and Sica, A. (2002). Macrophage polarization: tumor-associated macrophages as a paradigm for polarized M2 mononuclear phagocytes. Trends Immunol 23, 549-555.
Phillips, H. S., Kharbanda, S., Chen, R., Forrest, W. F., Soriano, R. H., Wu, T. D., Misra, A., Nigro, J. M., Colman, H., Soroceanu, L., et al. (2006). Molecular subclasses of high-grade glioma predict prognosis, delineate a pattern of disease progression, and resemble stages in neurogenesis. Cancer Cell 9, 157-173.
Piccirillo, S. G., Reynolds, B. A., Zanetti, N., Lamorte, G., Binda, E., Broggi, G., Brem, H., Olivi, A., Dimeco, F., and Vescovi, A. L. (2006). Bone morphogenetic proteins inhibit the tumorigenic potential of human brain tumour-initiating cells. Nature 444, 761-765.
Reya, T., Morrison, S. J., Clarke, M. F., and Weissman, I. L. (2001). Stem cells, cancer, and cancer stem cells. Nature 414, 105-111.
Ross, M. E., Mahfouz, R., Onciu, M., Liu, H. C., Zhou, X., Song, G., Shurtleff, S. A., Pounds, S., Cheng, C., Ma, J., et al. (2004). Gene expression profiling of pediatric acute myelogenous leukemia. Blood 104, 3679-3687.
Saadoun, S., Papadopoulos, M. C., Hara-Chikuma, M., and Verkman, A. S. (2005). Impairment of angiogenesis and cell migration by targeted aquaporin-1 gene disruption. Nature 434, 786-792.
St Croix, B., Rago, C., Velculescu, V., Traverso, G., Romans, K. E., Montgomery, E., Lal, A., Riggins, G. J., Lengauer, C., Vogelstein, B., et al. (2000). Genes expressed in human tumor endothelium. Science 289, 1197-1202.
Stupp, R., and Hegi, M. E. (2007). Targeting brain-tumor stem cells. Nat Biotechnol 25, 193-194. Stupp, R., Hegi, M. E., Gilbert, M. R., and Chakravarti, A. (2007). Chemoradiotherapy in Malignant Glioma: Standard of Care and Future Directions. J Clin Oncol 25, 4127-4136.
Stupp, R., Mason, W. P., van den Bent, M. J., Weller, M., Fisher, B., Taphoorn, M. J. B., Belanger, K., Brandes, A. A., Cairncross, J. G., Marosi, C., et al. (2005). Radiotherapy plus concomitant and adjuvant temozolomide for glioblastoma. N Engl J Med 352, 987-996.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550. Epub 12005 September 15530.
Vairapandi, M., Balliet, A. G., Hoffman, B., and Liebermann, D. A. (2002). GADD45b and GADD45g are cdc2/cyclinB1 kinase inhibitors with a role in S and G2/M cell cycle checkpoints induced by genotoxic stress. J Cell Physiol 192, 327-338.

1-32. (canceled)

33. A method for predicting or diagnosing outcome of concomitant chemo-radiotherapy of a subject suffering from brain tumor comprising:
(a) obtaining a biological sample from said subject,
(b) measuring the expression of gene clusters associated with tumor resistance to the concomitant chemo-radiotherapy treatment wherein said gene clusters are selected from the group comprising HOX genes, G13 genes, G18 genes, G25 genes, G07 genes and G14 genes, biologically active fragment thereof and/or combinations thereof,
(c) comparing the expression level of said gene clusters to threshold value, wherein the high expression of HOX genes, G13 genes, G18 genes and G25 genes indicate high risk for brain tumor resistance to the concomitant chemo-radiotherapy treatment, whereas the high expression of G07 genes and G14 genes indicate better outcome of the concomitant chemo-radiotherapy treatment, and
(d) optionally evaluating the medical prognosis of said subject based on the comparison of step (c), and/or adapting the treatment of said subject.

34. The method of claim 33, wherein the biological sample is a biopsy of brain tumor.

35. The method of claim 34, wherein the biopsy of brain tumor is a glioblastoma sample.

36. The method of claim 33, wherein the subject is a human.

37. The method of claim 33, wherein the HOX gene cluster includes one or more genes selected from the group comprising GADD45G, SCAP2, HOXD4, HOXC6, HOXA9, HOXA10, HOXA5, HOXA2, SCAP2, LOC400043, LOC375295, HOXD10, HOXD8, HOXA3, HOXA7, HOXD10, FLJ41747, PROM1, TSHZ2, and FAM110C

38. The method of claim 33, wherein the G13 gene cluster includes one or more genes selected from the group comprising B4GALNT1, AVIL, OS9, CDH1, CDK4, TSPAN31, METTL1, MDM2, CYP27B1, CPM, TSFM, FAM119B, SLC26A10, NUP107, KIAA1267, MGC5370, MARCH9, XRCC6BP1, DTX3, and RGS8.

39. The method of claim 33, wherein the G18 gene cluster includes one or more genes selected from the group comprising SLC1A3, ITPKB, MAOB, F3, BBOX1, DTNA, NDP, NR2E1, P2RY1, CA2, SLC7A11, AQP4, MLC1, CENTD1, SLC25A18, ITGB8, PAX6 and FLJ25530.

40. The method of claim 33, wherein the G25 gene cluster includes one or more genes selected from the group comprising EGFR, SOCS2, SEC61G, EYA2, SHOX2, EMILIN3, MASP1, FOXO1A, LHFP, and PDZD2.

41. The method of claim 33, wherein the G07 gene cluster includes one or more genes selected from the group comprising COL1 A1, KDELR2, LAMC1, COL6A3, LAMB1, LUM, COL3A1, NID1, VWF, LAMA4, MGP, SEC24D, COL1A2, PCOLCE, FMOD, FBN1, CD93, ADAM12, LOXL2, COL5A1, IGFBP6, KDELR3, TPM2, NID2, EDNRA, CDH5, LTBP2, ENPEP, SRPX2, ANGPT2, SERPINH1, PDLIM1, COL6A2, MXRA5, FN1, ANGPT2, COL13A1, FN1, COL4A2, COL4A1, NRP1, MYO1B, OLFML2B, SNAI2, PLXDC1, LXN, ELTD1 NOX4, COL5A2, ETS1, CTHRC1, MGC4677///L00541471, PELO, FAM20A, LOC493869, and GJA7.

42. The method of claim 33, wherein the G14 gene cluster includes one or more genes selected from the group comprising ALDH1A3, MME, THBS4, BMP5, MEOX1, COMP, GAS2, SEPT6///N-PAC, SOSTDC1, OLFML1, RP6-213H19.1, CYTL1, PRR16, TNMD, FNDC1, GLT8D2, CDC42EP5, SCARA5, COL12A1, DNM3, HMCN1 and MKX.

43. The method of claim 33, wherein measuring the expression of genes associated with tumor resistance to concomitant chemo-radiotherapy treatment is obtained by a method selected from the group consisting of:
(a) detecting RNA levels of said genes, and/or
(b) detecting a protein encoded by said genes, and/or
(c) detecting a biological activity of a protein encoded by said genes.

44. The method of claim 43, wherein the detecting of RNA levels is obtained through a technique selected from the group comprising Microarray hybridization, real-time polymerase chain reaction, Northern blot, In Situ Hybridization, sequencing-based methods, quantitative reverse transcription polymerase-chain reaction or RNAse protection assay.

45. The method of claim 43, wherein the detecting of protein levels is obtained through a technique selected from the group comprising Western blot, immunoprecipitation, immunohistochemistry, ELISA, Radio Immuno Assay, proteomics methods, or quantitative immunostaining methods.

46. A kit useful for predicting or diagnosing the tumor resistance in a subject treated with concomitant chemo-radiotherapy, said kit comprises a set of primers, probes or antibodies specific for one or more genes selected from gene clusters comprising HOX genes, G13 genes, G18 genes, G25 genes, G07 genes and G14 genes, biologically active fragment thereof and/or combinations thereof.

47. A method of treatment or prevention of tumor resistance in a subject suffering from a brain tumor comprising administering a therapeutically effective amount of modulator of expression of at least one gene selected from gene clusters comprising HOX genes, G13 genes, G18 genes, G25 genes, biologically active fragment thereof and/or combinations thereof.

48. The method of claim 47, wherein said modulator is inhibitor.

49. A pharmaceutical composition for the treatment or prevention of a tumor resistance in a subject suffering from a brain tumor, said composition comprising a pharmaceutically effective amount of modulator of expression of at least one gene selected from gene clusters comprising HOX genes, G13 genes, G18 genes, G25 genes, biologically active fragment thereof and/or combinations thereof.

50. A method for predicting or diagnosing a brain tumor in a subject, comprising:
(a) obtaining a biological sample from said subject,
(b) analyzing the epigenetic changes consisting in the promoter methylation of HOXA10 and HOXA9 gene in glioblastoma, wherein said epigenetic changes is associated with tumor malignancy

51. The method of claim 50, wherein the biological sample is body fluid, preferably cerebrospinal fluid (CSF) or blood.
